(19) United States
(12) Patent Application Publication
(10) Pub. No.: US 2016/0152725 A1
(43) Pub. Date: Jun. 2, 2016

(54) ANTIGEN-BINDING PROTEINS SPECIFIC FOR HLA-A2-RESTRICTED WILMS TUMOR 1 PEPTIDE

(71) Applicant: MEMORIAL SLOAN-KETTERING CANCER CENTER, New York, NY (US)

(72) Inventors: Nai-Kong V. Cheung, Purchase, NY (US); Mahiuddin Ahmed, New York, NY (US); Richard J. O'Reilly, Roxbury, CT (US); Dimiter V. Tassev, New York, NY (US); Qi Zhao, Hong Kong (CN)

(21) Appl. No.: 14/631,211

(22) Filed: Feb. 25, 2015

Related U.S. Application Data
(60) Provisional application No. 61/944,478, filed on Feb. 25, 2014.

(51) Int. Cl. C07K 16/32; C07K 16/30; A61K 51/10; C07K 16/46; A61K 47/48; A61K 49/00; C07K 16/28; C07K 14/725

(52) U.S. Cl. CPC C07K 16/32; C07K 16/2833; C07K 16/30; C07K 14/7051; C07K 16/468; A61K 47/48569; A61K 49/0058; A61K 51/1045; C07K 2317/565; C07K 2317/622; C07K 2317/55; C07K 2317/54; C07K 2319/30; C07K 2317/31; C07K 2317/92; C07K 2319/00; A61K 2039/505

(57) ABSTRACT

Antigen-binding proteins specific for HLA-A2-restricted Wilms tumor 1 peptide are disclosed. The antigen-binding proteins encompass antibodies in a variety of forms, including full-length antibodies, substantially intact antibodies, Fab fragments, F(ab′)2 fragments, and single chain Fv (scFv) fragments, as well as chimeric antigen receptors. Fusion proteins, such as scFv fusions with immunoglobulin or T-cell receptor domains, incorporating the antigen-binding proteins are provided. Methods of using the antigen-binding proteins in the treatment of hyperproliferative diseases such as cancer are also disclosed.

### CROSS-REFERENCE TO RELATED APPLICATION

The benefit under 35 U.S.C. §119(e) of U.S. Provisional Patent Application Ser. No. 61/944,478 filed Feb. 25, 2014, is hereby claimed, and the disclosure of the priority document is incorporated herein by reference in its entirety.

This application contains, as a separate part of the disclosure, a sequence listing in computer-readable form (filename: 48320_SeqListing; 33,469 bytes; created Feb. 24, 2015), which is incorporated by reference in its entirety.

### FIELD OF THE INVENTION

The present invention relates generally to antigen-binding molecules involved in immune function that are useful for cancer therapy.

### BACKGROUND OF THE INVENTION

Major histocompatibility complex (MHC) class I molecules play a central role in surveillance of aberrant or foreign proteins within cells. Peptides derived from endogenous proteins fill the MHC class I pockets and are recognized by T cell receptors (TCRs) on CD8(+) T lymphocytes (Doubrovina, E., et al., Blood, 2012, 120(8): p. 1633-46; Santomasso, B. D., et al., Proc Natl Acad Sci USA, 2007, 104(48): p. 19073-8). These MHC class I complexes are constitutively expressed by all nucleated cells. In cancer, virus-specific or tumor-specific peptide/MHC complexes represent a unique class of cell surface targets for immunotherapy, with promise in vaccine research (Keilholz, U., et al., Blood, 2009. 113(26): p. 6541-8), adoptive cell therapy (Yee, C., J Transl Med, 2005, 3(1): p. 17), and more recently, TCR-like antibodies (Dahan, R. and Y. Reiter, Expert Rev Mol Med, 2012, 14: p. e6; Cohen, C. J., et al., J Mol Recognit, 2003, 16(5): p. 324-32).

Although soluble TCRs have been successfully developed to target T cell epitopes (TCE) on tumors, their inherent low affinity has limited their potential as therapeutic reagents (Chames, P., et al., Proc Natl Acad Sci USA, 2000, 97(14): p. 7969-74). Even more importantly, the low density of MHC molecules and the individual peptides displayed by them further limits low-affinity reagents (Lev, A., et al., Proc Natl Acad Sci USA, 2004, 101(24): p. 9051-6). Attempts to increase affinity of TCRs using affinity maturation have been complicated by cross-reactivity (Stewart-Jones, G., et al., Proc Natl Acad Sci USA, 2009, 106(14): p. 5784-8; Holler, P. D. et al., Nat Immunol, 2003, 4(1): p. 55-62). Monoclonal antibodies are now an accepted modality in cancer treatment. Their GMP manufacture and downstream purification, as well as stability and formulation, have been optimized and standardized. Yet most, if not all, of these antibodies have targeted tumor antigens expressed on the tumor cell surface; and the repertoire of undiscovered cell surface antigens on solid tumors is rapidly shrinking.

TCR-like antibodies, with high affinity and controlled specificity, have the potential to be ideal therapeutics (Dahan, R. and Y. Reiter, Expert Rev Mol Med, 2012, 14: p. e6; Denkberg, G. and Y. Reiter, Autoimmun Rev, 2006, 5(4): p. 252-7). Several TCR-like antibodies targeting peptides derived from viral or tumor antigens in the context of human leukocyte antigen (HLA)-A1 or HLA-A2 have been described (Sastry, K. S., et al., J Virol, 2011, 85(5): p. 1935-42; Sergeeva, A., et al., Blood, 2011, 117(16): p. 4262-72; Verma, B., et al., J Immunol, 2010, 184(4): p. 2156-65; Willemsen, R. A., et al., Gene Ther, 2001, 8(21): p. 1601-8; Dao, T., et al., Sci Transl Med, 2013, 5(176): p. 176ra33; Tassev, D. V., M. Cheng, and N. K. Cheung, Cancer Gene Ther, 2012. 19(2): p. 84-100). One of the most studied tumor associated antigens has been the product of the Wilms tumor gene 1, which encodes a zinc-finger transcription factor (Wilms tumor protein 1; WT1) important in cell growth and differentiation (Renshaw, J., et al., Mol Cancer Ther, 2004, 3(11): p. 1467-84). The gene product is normally expressed in a tissue-specific manner, expressed mainly in the urogenital system of a developing embryo, as well as the central nervous and hematopoietic systems in adults (Yang, L., et al., Leukemia, 2007, 21(5): p. 868-76). In its aberrant state, WT1 expression has been linked to a variety of leukemias, lymphomas and solid tumors including astrocytic tumors, sarcomas, breast, lung and colorectal cancer, and neuroblastoma (Yang, L., et al., Leukemia, 2007, 21(5): p. 868-76; Sugiyama, H., Jpn J Clin Oncol, 2010, 40(5): p. 377-87). Although it was initially characterized as a tumor suppressor from studies with Wilms tumor, recent studies on WT1 in malignant cells has implicated its role as an oncogene (O'Reilly, R. J., et al., Semin Immunol, 2010, 22(3): p. 162-72).

Several peptides derived from endogenous WT1 protein are presented in the context of MHC class I molecules and are immunogenic (Doubrovina, E., et al., Blood, 2012, 120(8): p. 1633-46; Rezvani, K., et al., Clin Cancer Res, 2005, 11(24 Pt 1): p. 8799-807). The 9-mer WT1-derived peptide 126-134 having the amino acid sequence RMFPNAPYL (WT1₁₂₆), is the most extensively studied (Kohrt, H. E., et al., Blood, 2011, 118(19): p. 5319-29; Borbulevych, O. Y. et al., Mol Immunol, 2010, 47(15): p. 2519-24). Clinical studies involving the WT1₁₂₆ peptide have led to durable WT1-specific cytotoxic T cell (CTL) responses in cancers such as acute myeloid leukemia (AML) (Keilholz, U., et al., Blood, 2009. 113(26): p. 6541-8; Mailander, V., et al., Leukemia, 2004, 18(1): p. 165-6).

In addition to their targeting capabilities, TCR-like antibodies are highly useful reagents for studying specific antigen presentation on malignant and infected cells. Developing such antibodies and increasing their specificity for tumor-associated targets will facilitate the next generation of therapeutic antibodies.

### SUMMARY OF THE INVENTION

The present disclosure is directed to antigen-specific binding sequences from which a variety of antigen-binding proteins, fragments and derivatives thereof, and fusion proteins can be produced.

In one aspect, the disclosure provides an isolated antigen-binding protein or fragment or derivative thereof comprising one of: (A) an antigen-binding region comprising an amino acid sequence selected from the group consisting of SEQ ID NOs: 2 to 8 or 27; or (B) an antigen-binding region comprising a heavy chain variable domain (VH) and a light chain variable domain (VL), wherein the VH and VL, respectively, comprise amino acid sequences selected from the group consisting of SEQ ID NOs: (i) 9 and 13; (ii) 9 and 15; (iii) 11 and 16; (iv) 9 and 17; (v) 12 and 14; (vi) 9 and 14; and (vii) 10 and 13; or (C) an antigen-binding region comprising: (i) the following three heavy chain (HC) CDRs: (a) a HC CDR1 comprising the amino acid sequence set forth in SEQ ID NO: 18; and (b) a HC CDR2 comprising an amino acid sequence selected from the group consisting of SEQ ID NOs: 19 and 20; and (c) a HC CDR3 comprising the amino acid sequence set forth in SEQ ID NO: 21; and (ii) the following three light chain (LC) complementarity determining regions (CDRs): (a) a LC CDR1 comprising an amino acid sequence selected from the group consisting of SEQ ID NOs: 22 and 23; and (b) a LC CDR2 comprising an amino acid sequence selected from the group consisting of SEQ ID NOs: 24 and 25; and (c) a LC CDR3 comprising the amino acid sequence set forth in SEQ ID NO: 26.

For example, an isolated antigen-binding protein or fragment or derivative thereof according to the present disclosure may comprise one of: (A) an antigen-binding region comprising the amino acid sequence set forth in SEQ ID NO: 2; or (B) an antigen-binding region comprising a VH and a VL, wherein the VH and VL, respectively, comprise the amino acid sequences SEQ ID NOs: 9 and 13; or (C) an antigen-binding region comprising: (i) the following three HC CDRs: (a) a HC CDR1 comprising the amino acid sequence set forth in SEQ ID NO: 18; and (b) a HC CDR2 comprising the amino acid sequence set forth in SEQ ID NO: 20; and (c) a HC CDR3 comprising the amino acid sequence set forth in SEQ ID NO: 21; and (ii) the following three light chain (LC) CDRs: (a) a LC CDR1 comprising the amino acid sequence set forth in SEQ ID NO: 22; and (b) a LC CDR2 comprising the amino acid sequence set forth in SEQ ID NO: 25; and (c) a LC CDR3 comprising the amino acid sequence set forth in SEQ ID NO: 26. In one aspect, an isolated antigen-binding protein or fragment or derivative thereof according to the present disclosure comprises (i) the following three HC CDRs: (a) a HC CDR1 comprising the amino acid sequence set forth in SEQ ID NO: 18; and (b) a HC CDR2 comprising the amino acid sequence set forth in SEQ ID NO: 20; and (c) a HC CDR3 comprising the amino acid sequence set forth in SEQ ID NO: 21; and (ii) the following three light chain (LC) CDRs: (a) a LC CDR1 comprising the amino acid sequence set forth in SEQ ID NO: 22; and (b) a LC CDR2 comprising the amino acid sequence set forth in SEQ ID NO: 25; and (c) a LC CDR3 comprising the amino acid sequence set forth in SEQ ID NO: 26.

In one aspect, an isolated antigen-binding protein or fragment or derivative thereof according to the present disclosure has a heavy chain variable region comprising CDR1, CDR2, and CDR3 from SEQ ID NO: 9 and a light chain variable region comprising CDR1, CDR2, and CDR3 from SEQ ID NO: 13. In a further aspect, the antigen-binding protein or fragment or derivative thereof comprises a light chain variable region that is at least 90% identical to SEQ ID NO: 9, and a heavy chain variable region that is at least 90% identical to SEQ ID NO: 13, wherein the antigen-binding protein or fragment or derivative thereof is not Clone45.

In one aspect, an isolated antigen-binding protein of the present disclosure is an antibody. In one aspect, the antibody is a full-length antibody, a substantially intact antibody, or an antibody fragment, e.g., a Fab fragment, a F(ab′)2 fragment, or a single chain variable fragment (scFv). In another aspect, the isolated antigen-binding protein of the present disclosure is a chimeric antigen receptor (CAR). In one aspect, the disclosure provides an isolated scFv comprising a VH and a VL linked by an amino acid spacer, wherein the VH and VL, respectively, comprise amino acid sequences selected from the group consisting of SEQ ID NOs: (i) 9 and 13; (ii) 9 and 15; (iii) 11 and 16; (iv) 9 and 17; (v) 12 and 14; (vi) 9 and 14; and (vii) 10 and 13.

In one aspect, the present disclosure provides a fusion protein comprising an isolated antigen-binding protein or fragment or derivative thereof or scFV described herein. In one aspect, the fusion protein is a scFv-Fc fusion protein, an immunoconjugate, or a bispecific antibody. In one aspect, the fusion protein comprises a component selected from the group consisting of a cytotoxin, a detectable label, a radioisotope, a therapeutic agent, a nanoparticle, a liposome, a binding protein, or an antibody. In one aspect, the fusion protein comprises a binding protein or antibody having a binding specificity for a target that does not comprise WT1₁₂₆ (RMFPNAPYL; SEQ ID NO: 1). In one aspect, the fusion protein is a scFv-Fc fusion protein comprising a Fc from human IgG1. In one aspect, the fusion protein comprises the amino acid sequence set forth in SEQ ID NO: 27.

In one aspect, the present disclosure provides an isolated antigen-binding protein or fragment or derivative thereof, scFv, or fusion protein that specifically binds to an epitope on an HLA/peptide complex. In one aspect, the peptide of the HLA/peptide complex comprises the amino acid sequence set forth in SEQ ID NO: 1. Optionally, the HLA of the HLA/peptide complex is a MHC class I molecule, for example, a HLA-A2 molecule, such as HLA-A0201. In one aspect, the dissociation constant (K_{D}) of the antigen-binding protein or fragment or derivative thereof, scFv, or fusion protein to the HLA/peptide complex comprising the amino acid sequence set forth in SEQ ID NO: 1 is less than 60 nM, optionally less than 15 mM, less than 5 nM or less than 5 pM.

In one aspect, an isolated antigen-binding protein or fragment or derivative thereof, scFv, or fusion protein according to the present disclosure competes for binding to a target comprising WT₁₂₆ with an affinity-matured antibody.

In another aspect, the present disclosure provides a nucleic acid encoding an isolated antigen-binding protein or fragment or derivative thereof, isolated scFv, or fusion protein described herein. The present disclosure also provides an expression vector comprising the nucleic acid and a host cell transfected with the expression vector.

In another aspect, the present disclosure provides a pharmaceutical composition comprising an antigen-binding protein or fragment or derivative thereof, scFv, fusion protein, nucleic acid, expression vector, or host cell described herein, and a physiologically acceptable diluent, excipient, or carrier.

In one aspect, the present disclosure provides a cell expressing a chimeric antigen receptor (CAR) comprising an antigen-binding protein or fragment or derivative thereof or scFv described herein. In one aspect, the cell is a T cell or natural killer (NK) cell.

In another aspect, the present disclosure provides a method of diagnosing or treating a neoplastic, hyperplastic, or hyperproliferative disorder in a subject in need thereof comprising administering a therapeutically effective amount of an antigen-binding protein or fragment or derivative thereof, scFv, fusion protein, host cell, cell expressing a CAR, or pharmaceutical composition described herein. In one aspect, the present disclosure provides a method of inhibiting tumor growth or metastasis comprising contacting a tumor cell with an effective amount of an antigen-binding protein or fragment or derivative thereof, scFv, fusion protein, host cell, cell expressing a CAR, or pharmaceutical composition described herein. In one aspect, the present disclosure provides a method of diagnosing or treating cancer in a subject in need thereof comprising administering a therapeutically effective amount of an antigen-binding protein or fragment or derivative thereof, scFv, fusion protein, host cell, cell expressing a CAR, or pharmaceutical composition described herein. In one aspect, the methods disclosed herein further comprise administering a therapeutically effective amount of an effector cell and/or a cytokine.

In another aspect, the present disclosure provides a method of treatment comprising isolating T-cells from a subject, transfecting the T-cells with a vector comprising a nucleic acid encoding an isolated scFv described herein, such as an isolated scFv comprising a VH and a VL linked by an amino acid spacer, wherein the VH and VL, respectively, comprise amino acid sequences selected from the group consisting of SEQ ID NOS: (i) 9 and 13; (ii) 9 and 15; (iii) 11 and 16; (iv) 9 and 17; (v) 12 and 14; (vi) 9 and 14; and (vii) 10 and 13, and administering the transfected T-cells to the subject.

In another aspect, the present disclosure provides a kit comprising an antigen-binding protein or fragment or derivative thereof, scFv, fusion protein, nucleic acid, expression vector, host cell, cell expressing a CAR, or pharmaceutical composition described herein.

The foregoing summary is not intended to define every aspect of the invention, and other features and advantages of the present disclosure will become apparent from the following detailed description, including the drawings. The present disclosure is intended to be related as a unified document, and it should be understood that all combinations of features described herein are contemplated, even if the combination of features are not found together in the same sentence, paragraph, or section of this disclosure. In addition, the disclosure includes, as an additional aspect, all embodiments of the invention narrower in scope in any way than the variations specifically mentioned above. With respect to aspects of the disclosure described or claimed with “a” or “an,” it should be understood that these terms mean “one or more” unless context unambiguously requires a more restricted meaning. With respect to elements described as one or more within a set, it should be understood that all combinations within the set are contemplated. If aspects of the disclosure are described as “comprising” a feature, embodiments also are contemplated “consisting of” or “consisting essentially of” the feature. Additional features and variations of the disclosure will be apparent to those skilled in the art from the entirety of this application, and all such features are intended as aspects of the disclosure.

### BRIEF DESCRIPTION OF THE DRAWINGS

FIG. 1(A) and FIG. 1(B) show FACS for yeast display selection. FIG. 1(A): Sorting of yeast mutant library. Yeast library was labeled with mouse anti-c-myc antibody followed by fluorescent goat anti-mouse antibody, as well as biotinylated HLA-A2-WT1 monomer followed by fluorescent streptavidin (SA). During the three FACS selections, yeast cells were stained with decreasing concentrations of biotinylated HLA-A2-WT1 monomer at 100 μg/ml (sort1), 33 μg/ml (sort2) and 10 μg/ml (sort3), respectively. Each time, the brightest 0.1-0.3% cells were selected. FIG. 1(B): The binding and specificity of selected scFv-displayed yeast cells. Yeast cells of Clone45 or sort3 were stained with biotinylated HLA-A2-WT1 monomer (5 μg/ml) followed by fluorescent SA, PE conjugated HLA-A2-WT1 tetramer, or the negative control (HLA-A2-CDR2) tetramer followed by fluorescent goat-anti-mouse antibody.

FIG. 2(A) to FIG. 2(C) show ELISA of scFv variants and Q2L scFv-Fc against HLA-A2-peptide monomers. FIG. 2(A): Three scFvs (S3.1, S3.3 and S3.6) from the FACS sorting and parental Clone45 scFv were serially diluted and tested for binding to wells coated with HLA-A2-WT1 (RMFPNAPYL; SEQ ID NO: 1) monomer. FIG. 2(B): Q1L (single mutation), Q2L (double mutation) and S3.3 scFvs were serially diluted and added to wells coated with HLA-A2-WT1 (RMFPNAPYL; SEQ ID NO: 1) monomer. FIG. 2(C): The S3.1, S3.3, S3.6, Q2L parental scFvs and Q2L scFv-Fc were serially diluted and added to wells coated with WT1 peptide (RMFPNAPYL; SEQ ID NO: 1), three types of HLA-A2-WT1 monomers and four irrelevant HLA-A2 monomers. Bound scFv or scFv-Fc were detected with an HRP-conjugated anti-Flag tag antibody or HRP-conjugated anti-human Fc antibody and the optical densities (O.D.) at 490 nm after reaction with OIPD substrate were measured by Dynex MRX.

FIG. 3(A) and FIG. 3(B) show an overview of the WT1₁₂₆/HLA-A2 complex and structural modeling of TCR-like scFv. FIG. 3(A): Cross-eyed stereo view of the native WT1 peptide in the HLA-A2 peptide binding groove. The peptide N-terminus is to the right. The figure was adopted from Borbulevych et al., 2010, Mol Immunol. 47(15):2519-24. FIG. 3(B): Homology structure of parental Clone45 scFv was predicted by Rosetta software. When two glutamine residues at positions 50 of VH and 53 of VL were mutated, the affinity improved by 100-fold.

FIG. 4(A) and FIG. 4(B) show binding of TCR-like antibodies to WT1/HLA-A0201 complexes on live cells measured by flow cytometry. FIG. 4(A): Binding of Q2L scFv-Fc (right) and isotype matched TCR-like scFv-Fc (left) to T2 cells pulsed with WT1 peptide (dashed line), without peptide (solid line), or with irrelevant peptide (dotted line). T2 cells were then stained with TCR-like antibodies at 1 g/ml, followed by fluorescent secondary antibody. FIG. 4(B): Recognition of the naturally presented WT1/HLA-A2 complex on tumor cells by scFv variants. The human leukemia cells, THP-1 and BV173, were stained with scFvs at 10 μg/ml, followed by fluorescent secondary antibody.

FIG. 5(A) and FIG. 5(B) show ADCC of TCR-like antibodies against leukemia cells BA25 (FIG. 5(A)) and BV173 (FIG. 5(B)). Cytotoxicity of Q2L (diamonds), Clone45 (triangles) and isotype TCR-like scFv-Fc (squares) were measured by chromium release assay.

FIG. 6(A) to FIG. 6(E) show chimeric antigen receptor (CAR) expressing human lymphocytes specific for HLA-A2-WT1₁₂₆. FIG. 6(A): Schematic diagram of the CAR construct. The scFv sequence was cloned into the CAR gene and further cloned into a murine stem cell virus-based vector, which contained an internal ribosome entry site (IRES)-green fluorescence protein (GFP) sequence along with ampicillin-resistance. The resulting CAR was composed of the leader sequence, scFv and hinge region on the extracellular surface, a CD8α transmembrane domain, along with 4-1BB and the CD3ζ chain. FIG. 6(B): Transduced T cells derived from a single healthy donor. Both CD4 and CD8 T cells were genetically modified. CAR modified T cells were stained with HLA-A2-WT1₁₂₆ tetramer, anti-CD4, or anti-CD8 and analyzed by flow cytometry. FIG. 6(C): Specific cytotoxicity of Clone45-CAR (top) or Q2L-CAR (bottom) T cells against the tumor cell lines was measured by chromium release assay. FIG. 6(D): CAR NK-92 cells were stained with PE conjugated HLA-A2/WT1₁₂₆ tetramer (left) and two isotype controls: HLA-A2/Hud tetramer and HLA-A2/CDR2 tetramer. FIG. 6(E): Specific cytotoxicity of Q2L-CAR NK-92 (solid line) and mock (dashed line) cells against the tumor cell lines measured by chromium release assay.

FIG. 7(A) to FIG. 7(C) show that Q2L exhibits in vivo antitumor activity in a xenograft model of leukemia. Tumor burden was calculated by the luminescence signal of each mouse, and averaged (n=5 per group). Each scFv-Fc fusion antibody (Q2L, Clone45 or anti-HLA-A2/Hud [isotope control]) was administered intravenously twice a week for a total of 4 doses. FIG. 7(A): Q2L alone without human effectors significantly reduced tumor burden (p<0.05). FIG. 7(B) and FIG. 7(C): Human effectors (10 million per i.v. injection), and cytokine IL15/IL15α (10 g each s.c. injection) were given on day 7 and 14. Q2L was more effective compared to parental Clone45, in the absence (FIG. 7(B)) or presence (FIG. 7(C)) of IL15-IL15α. In contrast, the group treated with isotype control demonstrated rapid tumor growth.

FIG. 8 shows the amino acid sequences of the CDRs of the heavy and light chain and the scFv of Clone45.

FIG. 9 shows sensorgrams of binding kinetics of scFvs as measured with Biacore. Binding of scFv S3.1, S3.3, S3.6, Clone45 and Q2L are shown.

FIG. 10(A) and FIG. 10(B) show cells transfected with Q2L-CAR constructs.

FIG. 10(A): PG13 cells transfected with Q2L-CAR constructs were stained with WT1₁₂₆ tetramer. FIG. 10(B): K562 cells were transfected with Q2L-CAR and stained with WT1₁₂₆ tetramer.

FIG. 11(A) and FIG. 11(B) show epitope mapping. FIG. 11(A): Model of the docked complex of Q2L with the crystal structure of HLA-A2-WT1-RMF (pdb 3HPJ). The binding epitope was predicted to involve the heavy chain CDR2 of Q2L with Tyr 8 of WT1₁₂₆. FIG. 11(B): Binding of Q21 to T2 cells pulsed with WT1₁₂₆-wildtype (RMF), WT1₁₂₆-Arg1Ala (A1), WT1₁₂₆-Asn5Ala (A5) or WT1₁₂₆-Tyr8Ala (A8), measured by flow cytometry. A 40% reduction in binding was observed when Tyr8 was mutated to Ala. All peptides were verified to bind to HLA-A2 by staining with anti-HLA-A2 clone BB7.2.

### DETAILED DESCRIPTION OF THE INVENTION

The present invention provides compositions, kits, and methods of diagnosis and treatment relating to antigen-binding proteins that bind to a HLA-A2-restricted Wilms tumor 1 peptide. The antigen-binding proteins disclosed herein demonstrate improved specificity for an epitope comprising WT1₁₂₆ and can mediate tumor cell killing in vitro and in vivo.

Polypeptide sequences are indicated using standard three-letter abbreviations. Unless otherwise indicated, each polypeptide sequence has an amino terminus at the left and a carboxy terminus at the right. A particular polypeptide sequence also can be described by explaining how it differs from a reference sequence.

Unless otherwise defined herein, scientific and technical terms used in connection with the present invention shall have the meanings that are commonly understood by those of ordinary skill in the art. Further, unless otherwise required by context, singular terms shall include pluralities, and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art. In practicing the present invention, many conventional techniques in molecular biology, microbiology, cell biology, biochemistry, and immunology are used, which are within the skill of the art. Such techniques are described in greater detail in, for example, Molecular Cloning: a Laboratory Manual 3rd edition, J. F. Sambrook and D. W. Russell, ed. Cold Spring Harbor Laboratory Press 2001; Recombinant Antibodies for Immunotherapy, Melvyn Little, ed. Cambridge University Press 2009; “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001). The contents of these references and other references containing standard protocols, widely known to and relied upon by those of skill in the art, including manufacturers' instructions, are hereby incorporated by reference as part of the present disclosure.

The following abbreviations may be used throughout the disclosure and are generally intended to be interpreted consistently with the meaning of the terms as known in the art: ADCC: Antibody-dependent cellular cytotoxicity; ALL: Acute lymphocytic leukemia; AML: Acute myeloid leukemia; APC: Antigen presenting cell; β2M: Beta-2-microglobulin; BiTE: Bi-specific T cell engaging antibody; BLCL: EBV-transformed B-cell lymphoblastic cell line; CAR: Chimeric antigen receptor; CDC: Complement dependent cytotoxicity; CMC: Complement mediated cytotoxicity; CDR: Complementarity determining region (see also HVR below); CL: Constant domain of the light chain; CH₁: 1st constant domain of the heavy chain; CH_{1,2,3}: 1st, 2nd and 3rd constant domains of the heavy chain; CH_{2,3}: 2nd and 3rd constant domains of the heavy chain; CHO: Chinese hamster ovary; CTL: Cytotoxic T cell; EBNA3C: Epstein-Barr nuclear antigen 3C; EBV: Epstein-Barr virus; ECMV: Encephalomyocarditis virus; ER: Endoplasmic reticulum; E:T Ratio: Effector:Target ratio; Fab: Antibody binding fragment; FACS: Flow assisted cytometric cell sorting; FBS: Fetal bovine serum; GFP: Green fluorescence protein; HC: Heavy chain; HEL: Hen egg lysozyme; HLA: Human leukocyte antigen; HVR-H: Hypervariable region-heavy chain (see also CDR); HVR-L: Hypervariable region-light chain; Ig: Immunoglobulin; IPTG: isopropyl-1-thio-β-D-galactopyranoside; IRES: Internal ribosome entry site; K_{D}: Dissociation constant; k_{off}: Dissociation rate; kₒₙ: Association rate; MHC: Major histocompatibility complex; OPD: O-phenylenediamine; PEG: Polyethylene glycol; scFv: Single-chain variable fragment; SPR: Surface plasmon resonance; TB: Terrific Broth; TCE: T cell epitope; TCR: T cell receptor; TIL: Tumor infiltrating lymphocyte; VH: Variable heavy chain; VL: Variable light chain; and WT1: Wilms tumor protein 1.

In the description that follows, certain conventions will be followed regarding the usage of terminology. Generally, terms used herein are intended to be interpreted consistently with the meaning of those terms as described below and as they are known to those of skill in the art.

An “antigen-binding protein” is a protein or polypeptide that comprises an antigen-binding region or antigen-binding portion that has a strong affinity for another molecule to which it binds (antigen). Antigen-binding proteins encompass antibodies, antibody fragments, antibody derivatives, antibody analogs, fusion proteins, and antigen receptors including chimeric antigen receptors (CARs). An antigen-binding protein or fragment or derivative thereof optionally comprises a scaffold or framework portion that allows the antigen-binding portion to adopt a conformation that promotes binding of the antigen-binding protein to the antigen. The antigen-binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen-binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129 and Roque et al., 2004, Biotechnol. Prog. 20:639-654. In addition, peptide antibody mimetics (“PAMs”) can be used, as well as scaffolds based on antibody mimetics utilizing fibronectin components as a scaffold.

“Antibody” and “antibodies” refer to antigen-binding proteins that arise in the context of the immune system. The term “antibody” as referred to herein includes whole, full length antibodies and any fragment or derivative thereof in which the “antigen-binding portion” or “antigen-binding region” or single chains thereof are retained. A naturally occurring “antibody” (immunoglobulin) is a glycoprotein comprising at least two heavy (H) chains and two light (L) chains inter-connected by disulfide bonds. Each heavy chain is comprised of a heavy chain variable region (abbreviated herein as VH) and a heavy chain constant region. Heavy chains are classified as mu, delta, gamma, alpha, or epsilon, and define the antibody's isotype as IgM, IgD, IgG, IgA, and IgE, respectively. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The heavy and light chains form two regions: the Fab (fragment, antigen binding) region, also referred to as the variable (Fv) region, and the Fc (fragment, crystallizable) region. The variable regions (Fv) of the heavy and light chains contain a binding domain that interacts with an antigen. The constant (Fc) regions of the antibodies may mediate the binding to host tissues or factors, including various cells of the immune system (e.g., effector cells) and the first component (C1q) of the classical complement system. The term “Fc” as used herein includes native and mutein forms of polypeptides derived from the Fc region of an antibody. Truncated forms of such polypeptides containing the hinge region that promotes dimerization also are included. Fusion proteins comprising Fc moieties (and oligomers formed therefrom) offer the advantage of facile purification by affinity chromatography over Protein A or Protein G columns. One suitable Fc polypeptide is derived from the human IgG1 antibody.

Antibodies can be obtained from sources such as serum or plasma that contain immunoglobulins having varied antigenic specificity or recombinantly produced. If such antibodies are subjected to affinity maturation, they can be enriched for a particular antigenic specificity. Such affinity matured preparations of antibodies usually are made of less than about 10% of antibodies having specific binding activity for the particular antigen. Subjecting these preparations to several rounds of affinity maturation can increase the proportion of antibody having specific binding activity for the antigen. Antibodies prepared in this manner are often referred to as “affinity matured.”

Fragments, derivatives, or analogs of antigen-binding proteins such as antibodies can be readily prepared using techniques well-known in the art. The term “fragment” as used herein refers to a polypeptide that has an amino-terminal and/or carboxy-terminal deletion as compared to a corresponding full-length antigen-binding protein. Examples of fragments of antigen-binding proteins encompassed within the term “fragments” include a Fab fragment; a monovalent fragment consisting of the VL, VH, CL and CH1 domains; a F(ab′)2 fragment; a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989, Nature, 341:544-546), which consists of a VH domain; an isolated complementarity determining region (CDR); and a single chain variable fragment (scFv). A “derivative” of an antigen-binding protein is a polypeptide (e.g., an antibody) that has been chemically modified, e.g., via conjugation to another chemical moiety (such as, for example, polyethylene glycol or albumin, e.g., human serum albumin), phosphorylation, and/or glycosylation.

A “scFv” is a monovalent molecule that can be engineered by joining, using recombinant methods, the two domains of the Fv fragment, VL and VH, by a synthetic linker that enables them to be made as a single protein chain (see e.g., Bird et al., 1988, Science, 242:423-426; and Huston et al., 1988, Proc. Natl. Acad. Sci. 85:5879-5883). Such single chain antigen-binding peptides are also intended to be encompassed within the term “antigen-binding portion.” These antibody fragments are obtained using conventional techniques known to those of skill in the art, and the fragments are screened for utility in the same manner as are intact antibodies.

The term “antigen-binding portion” or “antigen-binding region” of an antigen-binding protein such as an antibody, as used herein, refers to that region or portion that confers antigen specificity; fragments of antigen-binding proteins, therefore, include one or more fragments of an antigen-binding protein that retain the ability to specifically bind to an antigen (e.g., an HLA-peptide complex). It has been shown that the antigen-binding function of an antibody can be performed by fragments of a full-length antibody.

An antigen-binding protein or fragment or derivative thereof or fusion protein thereof may have one or more binding sites. If there is more than one binding site, the binding sites may be identical to one another or may be different. For example, a naturally occurring human immunoglobulin typically has two identical binding sites, while a “bispecific antibody” or “bifunctional antibody” has two different binding sites.

An “epitope” is the portion of a molecule that is bound by an antigen-binding protein or fragment or derivative thereof (e.g., by an antibody). An epitope can comprise non-contiguous portions of the molecule, for example, in a polypeptide, amino acid residues that are not contiguous in the polypeptide's primary sequence, but that, in the context of the polypeptide's tertiary and quaternary structure, are near enough to each other to be bound by an antigen-binding protein).

The term “isolated” when referring to a molecule, for example, an antigen-binding protein or fragment or derivative thereof, is a molecule that by virtue of its origin or source of derivation (1) is not associated with naturally associated components that accompany it in its native state, (2) is substantially free of other molecules from the same species (3) is expressed by a cell from a different species, or (4) does not occur in nature without human intervention. In other words, an “isolated antigen-binding protein” or “isolated antibody” is one which has been identified and separated and/or recovered from a component of its natural environment. Thus, a molecule that is chemically synthesized, or synthesized in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

A “peptide,” “polypeptide” or “protein” is a molecule comprising two or more amino acid residues joined to each other by peptide bonds. These terms encompass, e.g., native and artificial proteins, protein fragments and polypeptide analogs (such as muteins, variants, and fusion proteins) of a protein sequence as well as post-translationally, or otherwise covalently or noncovalently, modified proteins. A peptide, polypeptide, or protein may be monomeric or polymeric.

A “conservative amino acid substitution” is one that does not substantially change the structural characteristics of the parent sequence (e.g., a replacement amino acid should not tend to break a helix that occurs in the parent sequence, or disrupt other types of secondary structure that characterize the parent sequence or are necessary for its functionality). Examples of art-recognized polypeptide secondary and tertiary structures are described in Proteins, Structures and Molecular Principles (Creighton, Ed., W.H. Freeman and Company, New York (1984)); Introduction to Protein Structure (C. Branden and J. Tooze, eds., Garland Publishing, New York, N.Y. (1991)); and Thornton et al. Nature, 1991, 354:105, which are each incorporated herein by reference.

The term “therapeutically effective” or “effective” depends on the condition of a subject and the specific peptide administered. The term refers to an amount effective to achieve a desired clinical effect. An effective amount varies with the nature of the condition being treated, the length of time that activity is desired, and the age and the condition of the subject, and ultimately is determined by the health care provider. In some aspects, an effective amount of an antigen-binding protein or fragment thereof according to the present disclosure is an amount effective to reduce or stop tumor growth.

Therapeutic antibodies have evolved in the past decade into an effective treatment for cancer. Although high-throughput proteomic profilings and bioinformatics tools have uncovered a large number of potential biomarkers in past decades (Rai, A. J., et al., Arch Pathol Lab Med, 2002, 126(12): p. 1518-26), most of these interesting tumor-specific markers are endogenous proteins inaccessible to current antibody therapy. Yet it is well known that peptides originating from intracellular proteins are presented on the surface of all nucleated cells, including tumor cells, by their MHC-I molecules. If specific antibodies can be made against these peptide-HLA complexes, a huge repertoire of targets is possible (Dahan, R. and Y. Reiter, Expert Rev Mol Med, 2012, 14: p. e6). In contrast to TCRs, where low affinity is an issue, TCR-like antibodies can be made to have high affinity while retaining specificity (Epel, M., et al., Eur J Immunol, 2008, 38(6): p. 1706-20). A number of TCR-like antibodies have been described that are directed against a large variety of MHC-class-I-peptide complexes derived from tumors as well as from pathogens (Dahan, R. and Y. Reiter, Expert Rev Mol Med, 2012, 14: p. e6; Denkberg, G. and Y. Reiter, Autoimmun Rev, 2006, 5(4): p. 252-7; Noy, R., et al., Expert Rev Anticancer Ther, 2005, 5(3): p. 523-36).

The present disclosure provides an algorithm for the discovery of TCR-like antigen-binding proteins directed toward an endogenous tumor-associated antigen, WT1, overexpressed by human malignant cells. In various embodiments, the antigen-binding proteins of the present disclosure bind to a conformational epitope of HLA-A2-restricted WT1₁₂₆ peptide, contacting the 126-134 residues of the WT1 protein (RMFPNAPYL; SEQ ID NO: 1). This epitope was previously validated as a tumor target recognizable by HLA-A2-restricted WT1₁₂₆ specific CD8⁺ T cells in patients with AML and CML (Rezvani, K., et al., Clin Cancer Res, 2005, 11(24 Pt 1): p. 8799-807). WT1 protein or mRNA expression has been described in leukemias and various types of solid cancers (Sugiyama, H., Jpn J Clin Oncol, 2010, 40(5): p. 377-87). A scFv having specificity for WT1 known as Clone45 was previously described (see U.S. Patent Publication No. 2014/0024809, and International Patent Application No. PCT/US2012/024885, incorporated herein by reference).

The evidence for naturally occurring high affinity TCR-like antibodies in human is scant. TCR-like antibodies were previously generated using hybridoma approaches (Sergeeva, A., et al., Blood, 2011, 117(16): p. 4262-72) or phage-display libraries (Engberg, J., et al., Methods Mol Biol, 2003, 207: p. 161-77). The affinity of TCR-like antibodies isolated from a human antibody phage-display library was relatively low and not always sufficient for therapeutic purposes (Sergeeva, A., et al., Blood, 2011, 117(16): p. 4262-72). The main mechanism for tumor evasion of T cell immunity is downregulation of HLA antigens (Gilham, D. E., et al., Trends Mol Med, 2012, 18(7): p. 377-84; Ramos, C. A. and G. Dotti, Expert Opin Biol Ther, 2011, 11(7): p. 855-73; Mardiros, A., et al., Blood, 2013, 122(18):3138-48). With a limited density of targets (either HLA-class I or the target peptide) on the cell surface, the affinity of a TCR-like antibody is critical.

In various embodiments, the present disclosure provides an efficient system for affinity maturation of antigen-binding proteins. The antigen-binding proteins, fragments and derivatives thereof, and fusion proteins of the present disclosure demonstrate high avidity binding to the TCE and to tumor targets and are capable of mediating antibody-dependent cell-mediated cytotoxicity or tumor lysis by chimeric antigen receptor (CAR) expressing human T or NK cells. The antigen-binding proteins, fragments and derivatives thereof, and fusion proteins of the present disclosure also demonstrate specific and potent cytotoxicity towards WT1-positive cancer cells that are HLA-A2 restricted in vivo.

In various embodiments, the present disclosure provides antigen-binding proteins, fragments and derivatives thereof, and fusion proteins comprising amino acid sequences described in Table 1.

TABLE 1

| | Sequence | SEQ ID NO |
|---|---|---|
| WT1₁₂₆ | RMFPNAPYL | 1 |
| Q2L scFV | EVQLLESGGGLVQPGGSLRLSCAASGFTFSSYAMSWVRQAP | 2 |
| | GKGLEWVSLIDPWGQETLYADSVKGRFTISRDNSKNTLYLQ | |
| | MNSLRAEDTAVYYCAKLTGRFDYWGQGTLVTVSSGGGGSG | |
| | GGGSGGGGSTDIQMTQSPSSLSASVGDRVTITCRASQSISSY | |
| | LNWYQQKPGKAPKLLIYSASLLQSGVPSRFSGSGSGTDFTLT | |
| | ISSLOPEDFATYYCOOGPGTPNTFGQGTKVEIKRA | |
| S3.3 scFV | EVQLLESGGGLVQPGGSLRLSCAASGFTFSSYAMSWVRQAP | 3 |
| | GKGLEWVSLIDPWGQETLYADSVKGRFTISRDNSKNTLYLQ | |
| | MNSLRAEDTAVYYCAKLTGRFDYWGQGTLVTVSSGGGGS | |
| | GGGGSGGGGSTNIQMTQSPSSLSASVGDRVTITCRASQSISS | |
| | YLNWYQQKPGKAPKLLIYSASLLQSGVPSRFSGNGSGTDFT | |
| | LTISSLQPEDFATYYCQQPGTPNTFGQGTKVEIKRA | |
| S3.1 scFV | EVQLLESGGGLVQPGGSLRLSCAASGFTFSSYAMSWVRQAP | 4 |
| | GKGLEWVSLIDPWGQETLYADSVKGRFTISRDNSKNTLYLQ | |
| | MNSLGAEDTAVYYCAKLTGRFDYWGQGTLVTVSSGGGGS | |
| | GGGGSGGGGSTDIQMTQSPSSLSASVGDRVTITCRASQSISS | |
| | YLNWYQQKPGKAPKLLIYSASQLQSGVPSRFSGSGSGTDFT | |
| | LTISNLQPEDFATYYCQQGPGTPNTFGQGTKVEIKRA | |
| S3.6 scFV | EVQLLESGGGLVQPGGSLRLSCAASGFTFSSYAMSWVRQAP | 5 |
| | GKGLEWVSLIDPWGQETLYADSVKGRFTISRDNSKNTLYLQ | |
| | MNSLRAEDTAVYYCAKLTGRFDYWGQGTLVTVSSGGGGS | |
| | GGGGSGGGGSTDIQMTQSPSSLSASVGDRVTITCRASQSISN | |
| | YLNWYQQKPGKAPKLLIYSASQLQSGVPSRFSGSGSGTVFT | |
| | LTISSLOPEDFATYYCOOGPGTPNTFGOGTKVEIKRA | |
| Q1L scFV | EVQLLESGGGLVQPGGSLRLSCAASGFTFSSYAMSWVRQAP | 6 |
| | GKGLEWVSLIDPWGQETLYADSVKGRFTISRDNSKNTLYLQ | |
| | MNSLRAEDTAVYYCAKLTGRFDYWGQGTLVTVSSGGGGS | |
| | GGGGSGGGGSTDIQMTQSPSSLSASVGDRVTITCRASQSISS | |
| | YLNWYQQKPGKAPKLLIYSASQLQSGVPSRFSGSGSGTDFT | |
| | LTISSLQPEDFATYYCQQGPGTPNTFGQGTKVEIKRA | |
| Q1aL scFV | EVQLLESGGGLVQPGGSLRLSCAASGFTFSSYAMSWVRQAP | 7 |
| | GKGLEWVSQIDPWGQETLYADSVKGRFTISRDNSKNTLYLQ | |
| | MNSLRAEDTAVYYCAKLTGRFDYWGQGTLVTVSSGGGGS | |
| | GGGGSGGGGSTDIQMTQSPSSLSASVGDRVTITCRASQSISS | |
| | YLNWYQQKPGKAPKLLIYSASLLQSGVPSRFSGSGSGTDFT | |
| | LTISSLQPEDFATYYCQQGPGTPNTFGQGTKVEIKRA | |
| S3.22 scFV | EVQLLESGGGLVQPGGSLRLSCAASGFLFSSYAMSWVRQAP | 8 |
| | GKGLEWVSLIDPWGQETLYADSVKGRFTISRDNSKNTLYLQ | |
| | MNSLRAEDTAVYYCAKLTGRFDYWGQGTLVTVSSGGGGS | |
| | GGGGSGGGGSTDIQMTQSPSSLSASVGDRVTITCRASQSISS | |
| | YLNWYQQKPGKAPKLLIYSASQLQSGVPSRFSGSGSGTDFT | |
| | LTISSLQPEDFATYYCQQGPGTPNTFGQGTKVEIKRA | |
| Q2L, S3.3, | EVQLLESGGGLVQPGGSLRLSCAASGFTFSSYAMSWVRQAP | 9 |
| S3.6,Q1L | GKGLEWVSLIDPWGQETLYADSVKGRFTISRDNSKNTLYLQ | |
| VH | MNSLRAEDTAVYYCAKLTGRFDYWGQGTLVTVSS | |
| Q1aL VH | EVQLLESGGGLVQPGGSLRLSCAASGFTFSSYAMSWVRQAP | 10 |
| | GKGLEWVSQIDPWGQETLYADSVKGRFTISRDNSKNTLYLQ | |
| | MNSLRAEDTAVYYCAKLTGRFDYWGQGTLVTVSS | |
| S3.1 VH | EVQLLESGGGLVQPGGSLRLSCAASGFTFSSYAMSWVRQAP | 11 |
| | GKGLEWVSLIDPWGQETLYADSVKGRFTISRDNSKNTLYLQ | |
| | MNSLGAEDTAVYYCAKLTGRFDYWGQGTLVTVSS | |
| S3.22 VH | EVQLLESGGGLVQPGGSLRLSCAASGFLFSSYAMSWVRQAP | 12 |
| | GKGLEWVSLIDPWGQETLYADSVKGRFTISRDNSKNTLYLQ | |
| | MNSLRAEDTAVYYCAKLTGRFDYWGQGTLVTVSS | |
| Q2L, | DIQMTQSPSSLSASVGDRVTITCRASQSISSYLNWYQQKPGK | 13 |
| Q1aL VL | APKLLIYSASLLQSGVPSRFSGSGSGTDFTLTISSLQPEDFAT | |
| | YYCQQGPGTPNTFGQGTKVEIKRA | |
| S3.22, | DIQMTQSPSSLSASVGDRVTITCRASQSISSYLNWYQQKPGK | 14 |
| Q1L VL | APKLLIYSASQLQSGVPSRFSGSGSGTDFTLTISSLQPEDFAT | |
| | YYCQQGPGTPNTFGQGTKVEIKRA | |
| S3.3 VL | NIQMTQSPSSLSASVGDRVTITCRASQSISSYLNWYQQKPGK | 15 |
| | APKLLIYSASLLQSGVPSRFSGNGSGTDFTLTISSLQPEDFAT | |
| | YYCQQGPGTPNTFGQGTKVEIKRA | |
| S3.1 VL | DIQMTQSPSSLSASVGDRVTITCRASQSISSYLNWYQQKPGK | 16 |
| | APKLLIYSASQLQSGVPSRFSGSGSGTDFTLTISNLQPEDFAT | |
| | YYCQQGPGTPNTFGQGTKVEIKRA | |
| S3.6 VL | DIQMTQSPSSLSASVGDRVTITCRASQSISNYLNWYQQKPGK | 17 |
| | APKLLIYSASQLQSGVPSRFSGSGSGTVFTLTISSLQPEDFAT | |
| | YYCQQGPGTPNTFGQGTKVEIKRA | |
| Q2L, S3.3, | SYAMS | 18 |
| S3.1, S3.6, | | |
| S3.22, | | |
| Q1L, Q1aL | | |
| HC CDR1 | | |
| Q1aL HC | QIDPWGQETLYADSVKG | 19 |
| CDR2 | | |
| Q2L, S3.3, | LIDPWGQETLYADSVKG | 20 |
| S.3.1, S3.6, | | |
| S3.22, Q1L | | |
| HC CDR2 | | |
| Q2L, S3.3, | LTGRFDY | 21 |
| S3.1, S.3.6, | | |
| S3.22, | | |
| Q1L, Q1aL | RASQSISSYLN | 22 |
| HC CDR3 | | |
| Q2L, S3.3, | | |
| S3.1, S3.22, | | |
| Q1L, Q1aL | | |
| LC CDR1 | | |
| S3.6 | RASQSISNYLN | 23 |
| LC CDR1 | | |
| S3.1, S3.6, | SASQLQS | 24 |
| S3.22, | | |
| Q1L LC | | |
| CDR2 | | |
| Q2L, S3.3, | SASLLQS | 25 |
| Q1aL | | |
| LC CDR2 | | |
| Q2L, S3.3, | QQGPGTPNT | 26 |
| S3.1, S.3.6, | | |
| S3.22, | | |
| Q1L, Q1aL | | |
| LC CDR3 | | |
| Q2L scFv-Fc | EVQLLESGGGLVQPGGSLRLSCAASGFTFSSYAMSWVRQAP | 27 |
| fusion | GKGLEWVSLIDPWGQETLYADSVKGRFTISRDNSKNTLYLQ | |
| protein | MNSLRAEDTAVYYCAKLTGRFDYWGQGTLVTVSSGGGGSG | |
| | GGGSGGGGSTDIQMTQSPSSLSASVGDRVTITCRASQSISSYL | |
| | NWYQQKPGKAPKLLIYSASLLQSGVPSRFSGSGSGTDFTLTIS | |
| | SLQPEDFATYYCQQGPGTPNTFGQGTKVEIKRKGPDKTHTCP | |
| | PCPAPELLGGPSVFLFPPKPKDTLMISRTPEVTCWVDVSHED | |
| | PEVKFNWYVDGVEVHNAKTKPREEQYNSTYRWSVLTVLH | |
| | QDWLNGKEYKCKVSNKALPAPIEKTISKAKGQPREPQVYTLP | |
| | PSRDELTKNQVSLTCLVKGFYPSDIAVEWESNGQPENNYKTT | |
| | PPVLDSDGSFFLYSKLTVDKSRWQQGNVFSCSVMHEALHNH | |
| | YTQKSLSLSPGK | |

The antigen-binding proteins, fragments and derivatives thereof, and fusion proteins of the present disclosure also include substantially homologous polypeptides that are 70%, 75%, 80%, 85%, 90%, 95%, 97%, 98%, or 99% identical to the peptides described in Table 1.

Antigen-binding proteins according to the present disclosure may be prepared by any of a number of conventional techniques. For example, they may be purified from cells that naturally express them (e.g., an antibody can be purified from a hybridoma that produces it), or produced in recombinant expression systems, using any technique known in the art. See, for example, Monoclonal Antibodies, Hybridomas: A New Dimension in Biological Analyses, Kennet et al. (eds.), Plenum Press, New York (1980); and Antibodies: A Laboratory Manual, Harlow and Land (eds.), Cold Spring Harbor Laboratory Press, Cold Spring Harbor, N.Y., (1988).

Any expression system known in the art can be used to make the recombinant polypeptides of the present disclosure. In general, host cells are transformed with a recombinant expression vector that comprises DNA encoding a desired polypeptide. Among the host cells that may be employed are prokaryotes, yeast or higher eukaryotic cells. Prokaryotes include gram negative or gram positive organisms, for example E. coli or bacilli. Higher eukaryotic cells include insect cells and established cell lines of mammalian origin. Examples of suitable mammalian host cell lines include the COS-7 line of monkey kidney cells (ATCC CRL 1651) (Gluzman et al., 1981, Cell 23:175), L cells, 293 cells, C127 cells, 3T3 cells (ATCC CCL 163), Chinese hamster ovary (CHO) cells, HeLa cells, BHK (ATCC CRL 10) cell lines, and the CVI/EBNA cell line derived from the African green monkey kidney cell line CVI (ATCC CCL 70) as described by McMahan et al., 1991, EMBO J. 10: 2821. Appropriate cloning and expression vectors for use with bacterial, fungal, yeast, and mammalian cellular hosts are described in the art, e.g., by Pouwels et al., Cloning Vectors: A Laboratory Manual, Elsevier, N. Y., 1985.

The transformed cells can be cultured under conditions that promote expression of the polypeptide, and the polypeptide recovered by conventional protein purification procedures. One such purification procedure includes the use of affinity chromatography, e.g., over a matrix having all or a portion of WT1 bound thereto. Polypeptides contemplated for use herein include substantially homogeneous recombinant mammalian anti-WT1₁₂₆ polypeptides substantially free of contaminating endogenous materials.

The amino acid sequence of the polypeptides disclosed herein may be verified by any means known in the art, and may be identical to the sequences disclosed herein in Table 1, or may differ from those sequences at one or more amino acid residues as result of processing. For example, on all or a portion of the substantially homogenous polypeptides, a C-terminal amino acid from either the light chain or the heavy chain (or relevant single-chain molecule) may be removed, by proteolytic processing or other processing that occurs during culture, for example, processing of C-terminal Lys residues. Alternatively, more than one C-terminal amino acid residue may be removed, for example two C-terminal amino acids, or three, four or five C-terminal amino acids. Similarly, N-terminal amino acids may be absent, for example, one, two, three, four or five N-terminal amino acids may be absent.

Alternatively, or additionally, the antigen-binding proteins, fragments and derivatives thereof, and fusion proteins of the present disclosure may undergo post-translational modifications, for example but not limited to, a glutamine may be cyclized or converted to pyroglutamic acid; additionally or alternatively, amino acids may undergo deamidation, isomerization, glycation and/or oxidation. The polypeptides of the invention may undergo additional post-translational modification, including glycosylation, for example N-linked or O-linked glycosylation, at sites that are well-known in the art. As described previously, changes may be made in the amino acid sequence of a polypeptide to preclude or minimize such alterations, or to facilitate them in circumstances where such processing is beneficial.

Antigen-binding polypeptides according to the present disclosure may be prepared, and screened for desired properties, by any of a number of known techniques. Certain of the techniques involve isolating a nucleic acid encoding a polypeptide chain (or portion thereof) of an antigen-binding protein of interest, and manipulating the nucleic acid through recombinant DNA technology. The nucleic acid may be fused to another nucleic acid of interest, or altered (e.g., by mutagenesis or other conventional techniques) to add, delete, or substitute one or more amino acid residues, for example.

Polypeptides of the present disclosure include polypeptides that have been modified in any way and for any reason, for example, to: (1) reduce susceptibility to proteolysis, (2) reduce susceptibility to oxidation, (3) alter binding affinity for forming protein complexes, (4) alter binding affinities, and (4) confer or modify other physicochemical or functional properties. Additionally, single or multiple amino acid substitutions (e.g., conservative amino acid substitutions) made in a sequence described in Table 1 (e.g., in the portion of the polypeptide outside the domain(s) forming intermolecular contacts) are encompassed by the present disclosure. Consensus sequences can be used to select amino acid residues for substitution; those of skill in the art recognize that additional amino acid residues may also be substituted.

Antigen-binding proteins (e.g., antibodies, antibody fragments, antibody derivatives, chimeric antigen receptors, and fusion proteins) of the invention can comprise any constant region known in the art. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. In one aspect, the light or heavy chain constant region is a fragment, derivative, variant, or mutein of a naturally occurring constant region.

In one aspect, the antigen-binding protein of the present invention comprises a fragment of an antibody. Such fragments can consist entirely of antibody-derived sequences or can comprise additional sequences. Fragments can be, for example, at least 5, 6, 7, 8, 9, 10, 11, 12, 13, 14, 15, 20, 50, 60, 70, 80, 90, 100, 150 or 200 amino acids in length. Fragments can also be, for example, at most 1,000, 750, 500, 250, 200, 175, 150, 125, 100, 90, 80, 70, 60, 50, 40, 30, 20, 15, 14, 13, 12, 11, or 10 amino acids in length. Fragments can also result from proteolytic (or other) processing, which, for example, results in variation in the amino and/or carboxy terminus of from one to five amino acids from that predicted. A fragment can further comprise, at either or both of its ends, one or more additional amino acids, for example, a sequence of amino acids from a different naturally-occurring protein (e.g., an Fc or leucine zipper domain) or an artificial amino acid sequence (e.g., an artificial linker sequence or a tag protein). Amino- and carboxy-termini of fragments or analogs may occur near boundaries of functional domains. Structural and functional domains can be identified by comparison of the nucleotide and/or amino acid sequence data to public or proprietary sequence databases. Computerized comparison methods can be used to identify sequence motifs or predicted protein conformation domains that occur in other proteins of known structure and/or function. Methods to identify protein sequences that fold into a known three-dimensional structure are known. See, e.g., Bowie et al., 1991, Science 253:164.

Examples of antigen-binding fragments include Fab, F(ab′)2, single chain antibodies such as scFvs, diabodies, triabodies, tetrabodies, and domain antibodies. Other examples are known in the art, e.g., as provided in Lunde et al., 2002, Biochem. Soc. Trans. 30:500-06.

In another aspect, the antigen-binding protein of the present invention comprises a derivative of an antibody. The derivative can comprise any molecule or substance that imparts a desired property, such as increased half-life in a particular use. Examples of molecules that can be used to form a derivative include, but are not limited to, albumin (e.g., human serum albumin) and polyethylene glycol (PEG). Albumin-linked and PEGylated derivatives of antibodies can be prepared using techniques well known in the art.

The present invention also provides non-peptide analogs of HLA-A2-restricted WT1-binding polypeptides. Non-peptide analogs are commonly used in the pharmaceutical industry as drugs with properties analogous to those of the template peptide. These types of non-peptide compound are termed “peptide mimetics” or “peptidomimetics,” see, for example, Fauchere, J. Adv. Drug Res. 15:29 (1986); Veber and Freidinger TINS p. 392 (1985); and Evans et al. J. Med. Chem. 30:1229 (1987), which are incorporated herein by reference. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a desired biochemical property or pharmacological activity), such as a human antibody, but have one or more peptide linkages optionally replaced by a linkage selected from the group consisting of: —CH₂NH—, —CH₂S—, —CH₂—CH₂—, —CH═—CH-(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may also be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992)), incorporated herein by reference).

In one aspect, the disclosure provides an isolated antigen-binding protein or fragment or derivative thereof comprising one of: (A) an antigen-binding region comprising an amino acid sequence selected from the group consisting of SEQ ID NOs: 2 to 8 or 27; or (B) an antigen-binding region comprising a heavy chain variable domain (VH) and a light chain variable domain (VL), wherein the VH and VL, respectively, comprise amino acid sequences selected from the group consisting of SEQ ID NOs: (i) 9 and 13; (ii) 9 and 15; (iii) 11 and 16; (iv) 9 and 17; (v) 12 and 14; (vi) 9 and 14; and (vii) 10 and 13; or (C) an antigen-binding region comprising: (i) the following three heavy chain (HC) CDRs: (a) a HC CDR1 comprising the amino acid sequence set forth in SEQ ID NO: 18; and (b) a HC CDR2 comprising an amino acid sequence selected from the group consisting of SEQ ID NOs: 19 and 20; and (c) a HC CDR3 comprising the amino acid sequence set forth in SEQ ID NO: 21; and (ii) the following three light chain (LC) complementarity determining regions (CDRs): (a) a LC CDR1 comprising an amino acid sequence selected from the group consisting of SEQ ID NOs: 22 and 23; and (b) a LC CDR2 comprising an amino acid sequence selected from the group consisting of SEQ ID NOs: 24 and 25; and (c) a LC CDR3 comprising the amino acid sequence set forth in SEQ ID NO: 26.

In one aspect, an isolated antigen-binding protein or fragment or derivative thereof according to the present disclosure comprises one of: (A) an antigen-binding region comprising the amino acid sequence set forth in SEQ ID NO: 2; or (B) an antigen-binding region comprising a VH and a VL, wherein the VH and VL, respectively, comprise the amino acid sequences SEQ ID NOs: 9 and 13; or (C) an antigen-binding region comprising: (i) the following three HC CDRs: (a) a HC CDR1 comprising the amino acid sequence set forth in SEQ ID NO: 18; and (b) a HC CDR2 comprising the amino acid sequence set forth in SEQ ID NO: 20; and (c) a HC CDR3 comprising the amino acid sequence set forth in SEQ ID NO: 21; and (ii) the following three light chain (LC) CDRs: (a) a LC CDR1 comprising the amino acid sequence set forth in SEQ ID NO: 22; and (b) a LC CDR2 comprising the amino acid sequence set forth in SEQ ID NO: 25; and (c) a LC CDR3 comprising the amino acid sequence set forth in SEQ ID NO: 26. In one aspect, an isolated antigen-binding protein or fragment or derivative thereof according to the present disclosure comprises (i) the following three HC CDRs: (a) a HC CDR1 comprising the amino acid sequence set forth in SEQ ID NO: 18; and (b) a HC CDR2 comprising the amino acid sequence set forth in SEQ ID NO: 20; and (c) a HC CDR3 comprising the amino acid sequence set forth in SEQ ID NO: 21; and (ii) the following three light chain (LC) CDRs: (a) a LC CDR1 comprising the amino acid sequence set forth in SEQ ID NO: 22; and (b) a LC CDR2 comprising the amino acid sequence set forth in SEQ ID NO: 25; and (c) a LC CDR3 comprising the amino acid sequence set forth in SEQ ID NO: 26.

In another aspect, an isolated antigen-binding protein or fragment or derivative thereof according to the present disclosure comprises one of: (A) an antigen-binding region comprising the amino acid sequence set forth in SEQ ID NO: 3; or (B) an antigen-binding region comprising a VH and a VL, wherein the VH and VL, respectively, comprise the amino acid sequences SEQ ID NOs: 9 and 15; or (C) an antigen-binding region comprising: (i) the following three HC CDRs: (a) a HC CDR1 comprising the amino acid sequence set forth in SEQ ID NO: 18; and (b) a HC CDR2 comprising the amino acid sequence set forth in SEQ ID NO: 20; and (c) a HC CDR3 comprising the amino acid sequence set forth in SEQ ID NO: 21; and (ii) the following three light chain (LC) CDRs: (a) a LC CDR1 comprising the amino acid sequence set forth in SEQ ID NO: 22; and (b) a LC CDR2 comprising the amino acid sequence set forth in SEQ ID NO: 25; and (c) a LC CDR3 comprising the amino acid sequence set forth in SEQ ID NO: 26.

In another aspect, an isolated antigen-binding protein or fragment or derivative thereof according to the present disclosure comprises one of: (A) an antigen-binding region comprising the amino acid sequence set forth in SEQ ID NO: 4; or (B) an antigen-binding region comprising a VH and a VL, wherein the VH and VL, respectively, comprise the amino acid sequences SEQ ID NOs: 11 and 16; or (C) an antigen-binding region comprising: (i) the following three HC CDRs: (a) a HC CDR1 comprising the amino acid sequence set forth in SEQ ID NO: 18; and (b) a HC CDR2 comprising the amino acid sequence set forth in SEQ ID NO: 20; and (c) a HC CDR3 comprising the amino acid sequence set forth in SEQ ID NO: 21; and (ii) the following three light chain (LC) CDRs: (a) a LC CDR1 comprising the amino acid sequence set forth in SEQ ID NO: 22; and (b) a LC CDR2 comprising the amino acid sequence set forth in SEQ ID NO: 24; and (c) a LC CDR3 comprising the amino acid sequence set forth in SEQ ID NO: 26.

In another aspect, an isolated antigen-binding protein or fragment or derivative thereof according to the present disclosure comprises one of: (A) an antigen-binding region comprising the amino acid sequence set forth in SEQ ID NO: 5; or (B) an antigen-binding region comprising a VH and a VL, wherein the VH and VL, respectively, comprise the amino acid sequences SEQ ID NOs: 9 and 17; or (C) an antigen-binding region comprising: (i) the following three HC CDRs: (a) a HC CDR1 comprising the amino acid sequence set forth in SEQ ID NO: 18; and (b) a HC CDR2 comprising the amino acid sequence set forth in SEQ ID NO: 20; and (c) a HC CDR3 comprising the amino acid sequence set forth in SEQ ID NO: 21; and (ii) the following three light chain (LC) CDRs: (a) a LC CDR1 comprising the amino acid sequence set forth in SEQ ID NO: 23; and (b) a LC CDR2 comprising the amino acid sequence set forth in SEQ ID NO: 24; and (c) a LC CDR3 comprising the amino acid sequence set forth in SEQ ID NO: 26.

In another aspect, an isolated antigen-binding protein or fragment or derivative thereof according to the present disclosure comprises one of: (A) an antigen-binding region comprising the amino acid sequence set forth in SEQ ID NO: 6; or (B) an antigen-binding region comprising a VH and a VL, wherein the VH and VL, respectively, comprise the amino acid sequences SEQ ID NOs: 9 and 14; or (C) an antigen-binding region comprising: (i) the following three HC CDRs: (a) a HC CDR1 comprising the amino acid sequence set forth in SEQ ID NO: 18; and (b) a HC CDR2 comprising the amino acid sequence set forth in SEQ ID NO: 20; and (c) a HC CDR3 comprising the amino acid sequence set forth in SEQ ID NO: 21; and (ii) the following three light chain (LC) CDRs: (a) a LC CDR1 comprising the amino acid sequence set forth in SEQ ID NO: 22; and (b) a LC CDR2 comprising the amino acid sequence set forth in SEQ ID NO: 24; and (c) a LC CDR3 comprising the amino acid sequence set forth in SEQ ID NO: 26.

In another aspect, an isolated antigen-binding protein or fragment or derivative thereof according to the present disclosure comprises one of: (A) an antigen-binding region comprising the amino acid sequence set forth in SEQ ID NO: 7; or (B) an antigen-binding region comprising a VH and a VL, wherein the VH and VL, respectively, comprise the amino acid sequences SEQ ID NOs: 10 and 13; or (C) an antigen-binding region comprising: (i) the following three HC CDRs: (a) a HC CDR1 comprising the amino acid sequence set forth in SEQ ID NO: 18; and (b) a HC CDR2 comprising the amino acid sequence set forth in SEQ ID NO: 19; and (c) a HC CDR3 comprising the amino acid sequence set forth in SEQ ID NO: 21; and (ii) the following three light chain (LC) CDRs: (a) a LC CDR1 comprising the amino acid sequence set forth in SEQ ID NO: 22; and (b) a LC CDR2 comprising the amino acid sequence set forth in SEQ ID NO: 25; and (c) a LC CDR3 comprising the amino acid sequence set forth in SEQ ID NO: 26.

In another aspect, an isolated antigen-binding protein or fragment or derivative thereof according to the present disclosure comprises one of: (A) an antigen-binding region comprising the amino acid sequence set forth in SEQ ID NO: 8; or (B) an antigen-binding region comprising a VH and a VL, wherein the VH and VL, respectively, comprise the amino acid sequences SEQ ID NOs: 12 and 14; or (C) an antigen-binding region comprising: (i) the following three HC CDRs: (a) a HC CDR1 comprising the amino acid sequence set forth in SEQ ID NO: 18; and (b) a HC CDR2 comprising the amino acid sequence set forth in SEQ ID NO: 20; and (c) a HC CDR3 comprising the amino acid sequence set forth in SEQ ID NO:21; and (ii) the following three light chain (LC) CDRs: (a) a LC CDR1 comprising the amino acid sequence set forth in SEQ ID NO: 22; and (b) a LC CDR2 comprising the amino acid sequence set forth in SEQ ID NO: 24; and (c) a LC CDR3 comprising the amino acid sequence set forth in SEQ ID NO: 26.

In one aspect, an isolated antigen-binding protein or fragment or derivative thereof according to the present disclosure comprises a heavy chain variable region comprising CDR1, CDR2, and CDR3 from a VH sequence in Table 1 and a light chain variable region comprising CDR1, CDR2, and CDR3 from a VL sequence in Table 1. For example, in one aspect, an isolated antigen-binding protein or fragment or derivative thereof according to the present disclosure comprises a heavy chain variable region comprising CDR1, CDR2, and CDR3 from SEQ ID NO: 9 and a light chain variable region comprising CDR1, CDR2, and CDR3 from SEQ ID NO: 13. In one aspect, an antigen-binding protein or fragment or derivative thereof according to the present disclosure comprises a heavy chain variable region comprising CDR1, CDR2, and CDR3 from a VH sequence in Table 1 that is at least 90% identical to that VH sequence and comprises a light chain variable region comprising CDR1, CDR2, and CDR3 from a VL sequence in Table 1 that is at least 90% identical to that VL sequence. For example, in one aspect, an antigen-binding protein or fragment or derivative thereof according to the present disclosure comprises a heavy chain variable region comprising CDR1, CDR2, and CDR3 from SEQ ID NO: 9 that is at least 90% identical to SEQ ID NO: 9 and comprises a light chain variable region comprising CDR1, CDR2, and CDR3 from SEQ ID NO: 13 that is at least 90% identical to SEQ ID NO: 13, wherein the antigen-binding protein is not Clone45.

In one aspect, an isolated antigen-binding protein of the present disclosure is an antibody. In one aspect, the antibody is a full-length antibody, a substantially intact antibody, or an antibody fragment, e.g., a Fab fragment, a F(ab′)2 fragment, or a single chain variable fragment (scFv). A scFv may be formed by linking heavy and light chain variable domain (Fv region) fragments via an amino acid bridge (short peptide linker), resulting in a single polypeptide chain. Such single-chain Fvs (scFvs) have been prepared by fusing DNA encoding a peptide linker between DNAs encoding the two variable domain polypeptides (VL and VH). The resulting polypeptides can fold back on themselves to form antigen-binding monomers, or they can form multimers (e.g., dimers, trimers, or tetramers), depending on the length of a flexible linker between the two variable domains (Kortt et al., 1997, Prot. Eng. 10:423; Kortt et al., 2001, Biomol. Eng. 18:95-108). By combining different V_{L} and V_{H}-comprising polypeptides, one can form multimeric scFvs that bind to different epitopes (Kriangkum et al., 2001, Biomol. Eng. 18:31-40). Techniques developed for the production of single chain antibodies include those described in U.S. Pat. No. 4,946,778; Bird, 1988, Science 242:423; Huston et al., 1988, Proc. Natl. Acad. Sci. USA 85:5879; Ward et al., 1989, Nature 334:544, de Graaf et al., 2002, Methods Mol. Biol. 178:379-87.

In another aspect, the isolated antigen-binding protein of the present disclosure is a chimeric antigen receptor (CAR).

In one aspect, the disclosure provides an isolated scFv comprising a VH and a VL linked by an amino acid spacer, wherein the VH and VL, respectively, comprise amino acid sequences selected from the group consisting of SEQ ID NOs: (i) 9 and 13; (ii) 9 and 15; (iii) 11 and 16; (iv) 9 and 17; (v) 12 and 14; (vi) 9 and 14; and (vii) 10 and 13. In one aspect, the amino acid spacer comprises serine and glycine residues.

In one aspect, the present disclosure provides a fusion protein comprising an isolated antigen-binding protein or scFV described herein. In one aspect, the fusion protein is a scFv-Fc fusion protein, an immunoconjugate, or a bispecific antibody. In one aspect, the fusion protein is a scFv-Fc fusion protein comprising a Fc from human IgG1. In one aspect, the fusion protein comprises the amino acid sequence set forth in SEQ ID NO: 27. The fusion protein can comprise, for example, a detectable (or labeling) moiety (e.g., a radioactive, colorimetric, antigenic or enzymatic molecule, a detectable bead (such as a magnetic or electrodense (e.g., gold) bead), or a molecule that binds to another molecule (e.g., biotin or streptavidin)), a therapeutic or diagnostic moiety (e.g., a radioactive, cytotoxic, or pharmaceutically active moiety), or a molecule that increases the suitability of the antibody for a particular use (e.g., administration to a subject, such as a human subject, or other in vivo or in vitro uses) such as a nanoparticle or liposome. In one aspect, the fusion protein comprises a component selected from the group consisting of a cytotoxin, a detectable label, a radioisotope, a therapeutic agent, a nanoparticle, a liposome, a binding protein, or an antibody.

In one aspect, the fusion protein comprises a binding protein or antibody having a binding specificity for a target that does not comprise SEQ ID NO: 1, e.g., a bi-specific or multi-specific antigen-binding protein. In one aspect, the fusion protein comprises a bispecific antibody that engages T cells.

Numerous methods of preparing bi- and multi-specific fusion proteins are known in the art. Such methods include the use of hybrid-hybridomas as described by Milstein et al., 1983, Nature 305:537, and others (U.S. Pat. No. 4,474,893, U.S. Pat. No. 6,106,833), and chemical coupling of antibody fragments (Brennan et al., 1985, Science, 229:81; Glennie et al., 1987, J. Immunol., 139:2367; U.S. Pat. No. 6,010,902). Moreover, bi- and multi-specific fusion proteins can be produced via recombinant means known in the art.

In one aspect, the present disclosure provides an isolated antigen-binding protein or fragment or derivative thereof, scFv, or fusion protein that specifically binds to an epitope on an HLA/peptide complex. In one aspect, the peptide of the HLA/peptide complex comprises the amino acid sequence RMFPNAPYL (SEQ ID NO: 1). In one aspect, the HLA of the HLA/peptide complex is a MHC class I molecule, optionally a HLA-A2 molecule, such as HLA-A0201 or another subtype. In one aspect, the dissociation constant (K_{D}) of the antigen-binding protein or fragment or derivative thereof, scFv, or fusion protein to the HLA/peptide complex comprising the amino acid sequence set forth in SEQ ID NO: 1 is less than 60 nM, less than 15 mM, less than 5 nM or less than 5 pM. The present disclosure provides antigen-binding proteins that exhibit an apparent binding affinity for a target comprising SEQ ID NO: 1 that is substantially the same as that of an antigen-binding protein or fragment or derivative thereof described herein in the Examples. In one aspect, an antigen-binding protein or fragment or derivative thereof, scFv, or fusion protein according to the present disclosure competes for binding to a target comprising SEQ ID NO: 1 with an affinity-matured antibody. In one aspect, an isolated antigen-binding protein or fragment or derivative thereof, scFv, or fusion protein described herein requires the tyrosine residue at position 8 of SEQ ID NO: 1 for high affinity binding.

In another aspect, the present disclosure provides a nucleic acid encoding the isolated antigen-binding protein or fragment or derivative thereof, isolated scFv, or fusion protein described herein. In one aspect, the nucleic acid encodes an isolated scFv comprising an amino acid sequence selected from the group consisting of SEQ ID NOs: 2 to 8, or an isolated scFv comprising a VH and a VL linked by an amino acid spacer, wherein the VH and VL, respectively, comprise amino acid sequences selected from the group consisting of SEQ ID NOS: (i) 9 and 13; (ii) 9 and 15; (iii) 11 and 16; (iv) 9 and 17; (v) 12 and 14; (vi) 9 and 14; and (vii) 10 and 13. The disclosure also provides an expression vector comprising a nucleic acid described herein, and a host cell transfected with an expression vector described herein. In one aspect, the host cell is a T-cell.

In another aspect, the present disclosure provides a pharmaceutical composition comprising an antigen-binding protein or fragment or derivative thereof, scFv, fusion protein, nucleic acid, expression vector, or host cell described herein, and a physiologically acceptable diluent, excipient, or carrier. Optionally, the composition additionally comprises one or more physiologically active agents, for example, a second inflammation- or immune-inhibiting substance, an anti-angiogenic substance, an analgesic substance, etc. In various particular embodiments, the composition comprises one, two, three, four, five, or six physiologically active agents in addition to an antigen-binding protein or fragment or derivative thereof, scFv, fusion protein, nucleic acid, expression vector, or host cell.

In one aspect, a pharmaceutical composition of the present disclosure comprises an antigen-binding protein or fragment or derivative thereof described herein with one or more substances selected from the group consisting of a buffer, an antioxidant such as ascorbic acid, a low molecular weight polypeptide (such as those having fewer than 10 amino acids), a protein, an amino acid, a carbohydrate such as glucose, sucrose or dextrins, a chelating agent such as EDTA, glutathione, a stabilizer, and an excipient. Neutral buffered saline or saline mixed with conspecific serum albumin are examples of appropriate diluents. In accordance with appropriate industry standards, preservatives such as benzyl alcohol may also be added. The composition may be formulated as a lyophilizate using appropriate excipient solutions (e.g., sucrose) as diluents. Suitable components are nontoxic to recipients at the dosages and concentrations employed. Further examples of components that may be employed in pharmaceutical formulations are presented in Remington's Pharmaceutical Sciences, 16^{th} Ed. (1980) and 20^{th} Ed. (2000), Mack Publishing Company, Easton, Pa.

As is understood in the art, pharmaceutical compositions comprising the molecules of the present disclosure are administered to a subject in a manner appropriate to the indication. A pharmaceutical composition of the present disclosure comprising an antigen-binding protein or fragment or derivative thereof, scFv, fusion protein, or cell expressing a CAR described herein may be formulated for delivery by any route that provides an effective dose of the immunogen. Pharmaceutical compositions may be administered by any suitable technique, including but not limited to parenterally, topically, or by inhalation. If injected, the pharmaceutical composition can be administered, for example, via intra-articular, intravenous, intramuscular, intralesional, intraperitoneal or subcutaneous routes, by bolus injection, or continuous infusion. Localized administration, e.g. at a site of disease or injury is contemplated, as are transdermal delivery and sustained release from implants. Delivery by inhalation includes, for example, nasal or oral inhalation, use of a nebulizer, inhalation of the antagonist in aerosol form, and the like. Other alternatives include eyedrops; oral preparations including tablets, capsules, syrups, lozenges or chewing gum; and topical preparations such as lotions, gels, sprays, patches, and ointments.

In one aspect, the present disclosure provides a cell expressing a chimeric antigen receptor (CAR) comprising an antigen-binding protein or fragment or derivative thereof or scFv described herein. In one aspect, the cell is a T cell or natural killer (NK) cell. Methods for producing a cell expressing a CAR are provided in the examples and known in the art.

In another aspect, the present disclosure provides a method of diagnosing or treating a neoplastic, hyperplastic, or hyperproliferative disorder in a subject in need thereof comprising administering a therapeutically effective amount of an antigen-binding protein or fragment or derivative thereof, scFv, fusion protein, nucleic acid, expression vector, host cell, cell expressing a CAR, or pharmaceutical composition described herein. In one aspect, the present disclosure provides a method of inhibiting tumor growth or metastasis comprising contacting a tumor cell with an effective amount of an antigen-binding protein or fragment or derivative thereof, scFv, fusion protein, nucleic acid, expression vector, host cell, cell expressing a CAR, or pharmaceutical composition described herein.

In one aspect, the present disclosure provides a method of diagnosing or treating cancer in a subject in need thereof comprising administering a therapeutically effective amount of an antigen-binding protein or fragment or derivative thereof, scFv, fusion protein, cell expressing a CAR, or pharmaceutical composition described herein.

In another aspect, the present disclosure provides a method of treatment comprising isolating T-cells from a subject, transfecting the T-cells with an expression vector comprising a nucleic acid encoding an isolated antigen-binding protein or fragment or derivative thereof described herein, and administering the transfected T-cells to the subject. In one aspect, the expression vector comprises a nucleic acid encoding an isolated scFv comprising a VH and a VL linked by an amino acid spacer, wherein the VH and VL, respectively, comprise amino acid sequences selected from the group consisting of SEQ ID NOS: (i) 9 and 13; (ii) 9 and 15; (iii) 11 and 16; (iv) 9 and 17; (v) 12 and 14; (vi) 9 and 14; and (vii) 10 and 13.

In one aspect, the cancer is selected from the group consisting of adrenal cancer, acinic cell carcinoma, acoustic neuroma, acral lentigious melanoma, acrospiroma, acute eosinophilic leukemia, acute erythroid leukemia, acute lymphoblastic leukemia, acute megakaryoblastic leukemia, acute monocytic leukemia, acute myeloid/myelogenous leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, B-cell chronic lymphocytic leukemia, B-cell prolymphocytic leukemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, chronic myelocytic leukemia, clear-cell sarcoma of the kidney, craniopharyngioma, cutaneous T-cell lymphoma, cervical cancer, colon cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, Ewing's sarcoma, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma, glioma, gliomatosis cerebri, glucagonoma, gonadoblastoma, granulosa cell tumor, gynandroblastoma, gallbladder cancer, gastric cancer, gastrointestinal cancer, hairy cell leukemia, hemangioblastoma, head and neck cancer, hemangiopericytoma, hematological malignancy, hepatoblastoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, chronic lymphocytic leukemia, liver cancer, small cell lung cancer, non-small cell carcinoma, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myelodysplastic syndrome, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, preimary peritoneal cancer, prostate cancer, pancreatic cancer, pharyngeal cancer, pseudomyxoma periotonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, and Wilms tumor.

In one aspect, the methods disclosed herein further comprise administering a therapeutically effective amount of an effector cell and/or a cytokine. In one aspect, the cytokine interleukin 15 (IL-15) is administered.

The methods of treatment of the present disclosure encompass alleviation or prevention of at least one symptom or other aspect of a disorder, or reduction of disease severity, and the like. An antigen-binding protein or fragment or derivative thereof, scFv, fusion protein, nucleic acid, expression vector, host cell, cell expressing a CAR, or pharmaceutical composition described herein need not effect a complete cure, or eradicate every symptom or manifestation of a disease, to constitute a viable therapeutic agent. As is recognized in the art, therapeutic agents may reduce the severity of a given disease state, but need not abolish every manifestation of the disease to be regarded as useful. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient.

Dosages and the frequency of administration for use in the methods of the present disclosure may vary according to such factors as the route of administration, the particular antigen-binding proteins employed, the nature and severity of the disease to be treated, whether the condition is acute or chronic, and the size and general condition of the subject. Appropriate dosages can be determined by procedures known in the pertinent art, e.g. in clinical trials that may involve dose escalation studies.

An antigen-binding protein or fragment or derivative thereof, scFv, fusion protein, nucleic acid, expression vector, host cell, cell expressing a CAR, or pharmaceutical composition of the present disclosure may be administered, for example, once or more than once, e.g., at regular intervals over a period of time. In various embodiments, time interval between administration of doses of the antigen-binding protein or fragment or derivative thereof, scFv, fusion protein, nucleic acid, expression vector, host cell, cell expressing a CAR, or pharmaceutical composition may be at least one, two, three, four, five, six, or seven days or one, two, three, four, five, six, seven, or eight weeks, or may be at least one, two, three, four, five, six, seven, eight, nine, ten, or eleven months, or at least one, two, three, or four years. In general, the antigen-binding protein or fragment or derivative thereof, scFv, fusion protein, nucleic acid, expression vector, host cell, cell expressing a CAR, or pharmaceutical composition is administered to a subject until the subject manifests a medically relevant degree of improvement over baseline for the chosen indicator or indicators.

In general, the amount of an antigen-binding protein or fragment or derivative thereof, scFv, or fusion protein described herein present in a dose, or produced in situ by an encoding polynucleotide present in a dose, ranges from about 0.01 μg to about 1000 μg per kg of host. In one aspect, cells expressing a CAR, may be administered at a dose of 1.5×10⁶ to 3.0×10⁶ CAR-expressing cells/kg. Other host cells may also be administered at a dose of 1.5×10⁶ to 3.0×10⁶ cells/kg. The use of the minimum dosage that is sufficient to provide effective therapy is usually preferred. Patients may generally be monitored for therapeutic or prophylactic effectiveness using assays suitable for the condition being treated or prevented, which assays will be familiar to those having ordinary skill in the art and which are described herein. The methods disclosed herein may include oral administration of an antigen-binding protein or fragment or derivative thereof, scFv, or fusion protein or delivery by injection of a liquid pharmaceutical composition. A liquid pharmaceutical composition may include, for example, one or more of the following: a sterile diluent such as water for injection, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. The use of physiological saline is preferred, and an injectable pharmaceutical composition is preferably sterile. When administered in a liquid form, suitable dose sizes will vary with the size of the subject, but will typically range from about 1 ml to about 500 ml (comprising from about 0.01 μg to about 1000 μg per kg) for a 10-60 kg subject. Optimal doses may generally be determined using experimental models and/or clinical trials. The optimal dose may depend upon the body mass, body area, weight, or blood volume of the subject. As described herein, the appropriate dose may also depend upon the patient's (e.g., human) condition, that is, stage of the disease, general health status, as well as age, gender, and weight, and other factors familiar to a person skilled in the medical art.

In particular embodiments of the methods described herein, the subject is a human or non-human animal. A subject in need of the treatments described herein may exhibit symptoms or sequelae of a disease, disorder, or condition described herein or may be at risk of developing the disease, disorder, or condition. Non-human animals that may be treated include mammals, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

In another aspect, the present disclosure provides a kit comprising an antigen-binding protein or fragment or derivative thereof, scFv, fusion protein, nucleic acid, expression vector, host cell, cell expressing a CAR, or pharmaceutical composition described herein. Kits for use by medical practitioners include an antigen-binding polypeptide of the invention and a label or other instructions for use in treating any of the conditions discussed herein. Instructions typically describe methods for administration, including methods for determining the proper state of the subject, the proper dosage amount, and the proper administration method, for administering the composition. Instructions can also include guidance for monitoring the subject over the duration of the treatment time. Kits provided herein also can include devices for administration of a pharmaceutical composition described herein to a subject. Any of a variety of devices known in the art for administering medications, immunogenic compositions, and vaccines can be included in the kits provided herein. Exemplary devices include, but are not limited to, a hypodermic needle, an intravenous needle, a catheter, a needle-less injection device, an inhaler, and a liquid dispenser, such as an eyedropper. Typically, the device for administering a composition is compatible with the active components of the kit.

Embodiments contemplated in view of the foregoing description include, but are not limited to, the following numbered embodiments:

1. An isolated antigen-binding protein or fragment or derivative thereof comprising one of: (A) an antigen-binding region comprising an amino acid sequence selected from the group consisting of SEQ ID NOs: 2 to 8 or 27; or (B) an antigen-binding region comprising a heavy chain variable domain (VH) and a light chain variable domain (VL), wherein the VH and VL, respectively, comprise amino acid sequences selected from the group consisting of SEQ ID NOs: (i) 9 and 13; (ii) 9 and 15; (iii) 11 and 16; (iv) 9 and 17; (v) 12 and 14; (vi) 9 and 14; and (vii) 10 and 13; or (C) an antigen-binding region comprising: (i) the following three heavy chain (HC) CDRs: (a) a HC CDR1 comprising the amino acid sequence set forth in SEQ ID NO: 18; and (b) a HC CDR2 comprising an amino acid sequence selected from the group consisting of SEQ ID NOs: 19 and 20; and (c) a HC CDR3 comprising the amino acid sequence set forth in SEQ ID NO: 21; and (ii) the following three light chain (LC) complementarity determining regions (CDRs): (a) a LC CDR1 comprising an amino acid sequence selected from the group consisting of SEQ ID NOs: 22 and 23; and (b) a LC CDR2 comprising an amino acid sequence selected from the group consisting of SEQ ID NOs: 24 and 25; and (c) a LC CDR3 comprising the amino acid sequence set forth in SEQ ID NO: 26.

2. The isolated antigen-binding protein or fragment or derivative thereof of embodiment 1 comprising one of: (A) an antigen-binding region comprising the amino acid sequence set forth in SEQ ID NO: 2; or (B) an antigen-binding region comprising a heavy chain variable domain (VH) and a light chain variable domain (VL), wherein the VH and VL, respectively, comprise the amino acid sequences SEQ ID NOs: 9 and 13; or (C) an antigen-binding region comprising: (i) the following three heavy chain (HC) complementarity determining regions (CDRs): (a) a HC CDR1 comprising the amino acid sequence set forth in SEQ ID NO: 18; and (b) a HC CDR2 comprising the amino acid sequence set forth in SEQ ID NO: 20; and (c) a HC CDR3 comprising the amino acid sequence set forth in SEQ ID NO: 21; and (ii) the following three light chain (LC) CDRs: (a) a LC CDR1 comprising the amino acid sequence set forth in SEQ ID NO: 22; and (b) a LC CDR2 comprising the amino acid sequence set forth in SEQ ID NO: 25; and (c) a LC CDR3 comprising the amino acid sequence set forth in SEQ ID NO: 26.

3. An isolated antigen-binding protein or fragment or derivative thereof having a heavy chain variable region comprising CDR1, CDR2 and CDR3 from SEQ ID NO: 9, and a light chain variable region comprising CDR1, CDR2 and CDR3 from SEQ ID NO: 13.

4. The isolated antigen-binding protein or fragment or derivative thereof of embodiment 3, wherein the light chain variable region is at least 90% identical to SEQ ID NO: 9, and the heavy chain variable region is at least 90% identical to SEQ ID NO: 13; and wherein the antigen-binding protein is not Clone45.

5. The isolated antigen-binding protein or fragment or derivative thereof of any of embodiments 1-4, wherein the isolated antigen-binding protein is an antibody.

6. The isolated antigen-binding protein or fragment or derivative thereof of embodiment 5, wherein the antibody is a full-length antibody, a substantially intact antibody, a Fab fragment, a F(ab′)2 fragment, or a single chain variable fragment (scFv).

7. The isolated antigen-binding protein or fragment or derivative thereof of embodiment 6, wherein the antibody is a scFv.

8. The isolated antigen-binding protein or fragment or derivative thereof of embodiment 1, wherein the isolated antigen-binding protein or fragment or derivative thereof is a chimeric antigen receptor (CAR).

9. An isolated scFv comprising a VH and a VL linked by an amino acid spacer, wherein the VH and VL, respectively, comprise amino acid sequences selected from the group consisting of SEQ ID NOs: (i) 9 and 13; (ii) 9 and 15; (iii) 11 and 16; (iv) 9 and 17; (v) 12 and 14; (vi) 9 and 14; and (vii) 10 and 13.

10. A fusion protein comprising the antigen-binding protein or fragment or derivative thereof or scFv of any of embodiments 1-9.

11. The fusion protein of embodiment 10, wherein the fusion protein comprises a scFv-Fc fusion protein, immunoconjugate, or bispecific antibody.

12. The fusion protein of embodiment 10 or 11, wherein the fusion protein comprises the amino acid sequence set forth in SEQ ID NO: 27.

13. The fusion protein of any of embodiments 10-12, wherein the fusion protein comprises a bispecific antibody that engages T cells.

14. The fusion protein of any of embodiments 10-13, wherein the fusion protein comprises a second component selected from the group consisting of a cytotoxin, a detectable label, a radioisotope, a therapeutic agent, a liposome, a nanoparticle, a binding protein, or an antibody.

15. The fusion protein of any of embodiments 10-14, wherein the fusion protein is a scFv-Fc fusion protein comprising a Fc from human IgG1.

16. The fusion protein of any of embodiments 10-15, wherein the fusion protein comprises a binding protein or antibody having a binding specificity for a target that does not comprise the amino acid sequence set forth in SEQ ID NO: 1.

17. The isolated antigen-binding protein or fragment or derivative thereof, isolated scFv, or fusion protein of any of embodiments 1-16, wherein the antigen-binding protein or fragment or derivative thereof, scFv, or fusion protein specifically binds to an epitope on a HLA/peptide complex.

18. The isolated antigen-binding protein or fragment or derivative thereof, isolated scFv, or fusion protein of embodiment 17, wherein the peptide of the HLA/peptide complex comprises the amino acid sequence set forth in SEQ ID NO: 1.

19. The isolated antigen-binding protein or fragment or derivative thereof, isolated scFv, or fusion protein of embodiment 17 or 18, wherein the HLA of the HLA/peptide complex is a MHC class I molecule.

20. The isolated antigen-binding protein or fragment or derivative thereof, isolated scFv, or fusion protein of embodiment 19, wherein the MHC class I molecule is a HLA-A2 molecule.

21. The isolated antigen-binding protein or fragment or derivative thereof, isolated scFv, or fusion protein of embodiment 20, wherein the HLA-A2 molecule is HLA-A0201.

22. The isolated antigen-binding protein or fragment or derivative thereof, isolated scFv, or fusion protein of any of embodiments 17-21, wherein the dissociation constant (K_{D}) of the antigen-binding protein or fragment or derivative thereof, scFv, or fusion protein to the HLA/peptide complex is less than 60 nM, optionally less than 15 nM or less than 5 nM or less than 6 pM.

23. The isolated antigen-binding protein or fragment or derivative thereof, isolated scFv, or fusion protein of any of embodiments 18-22, wherein the isolated antigen-binding protein or fragment or derivative thereof, scFv, or fusion protein requires a tyrosine residue at position 8 of SEQ ID NO: 1 for high affinity binding.

24. The isolated antigen-binding protein or fragment or derivative thereof, scFv, or fusion protein of any of embodiments 1-23, wherein the antigen-binding protein or fragment or derivative thereof, scFv, or fusion protein competes for binding to WT1₁₂₆ with an affinity-matured antibody.

25. A nucleic acid encoding the isolated antigen-binding protein or fragment or derivative thereof, isolated scFv, or fusion protein of any of embodiments 1-24.

26. The nucleic acid of embodiment 25, wherein the nucleic acid encodes an isolated scFv comprising a VH and a VL linked by an amino acid spacer, wherein the VH and VL, respectively, comprise amino acid sequences selected from the group consisting of SEQ ID NOs: (i) 9 and 13; (ii) 9 and 15; (iii) 11 and 16; (iv) 9 and 17; (v) 12 and 14; (vi) 9 and 14; and (vii) 10 and 13.

27. An expression vector comprising the nucleic acid of embodiment 25 or 26.

28. A host cell transfected with the expression vector of embodiment 27.

29. The host cell of embodiment 28, wherein the host cell is a T-cell.

30. A pharmaceutical composition comprising an antigen-binding protein or fragment or derivative thereof, scFv, fusion protein, nucleic acid, expression vector, or host cell of any of embodiments 1-29 and a physiologically acceptable diluent, excipient or carrier.

31. A cell expressing a chimeric antigen receptor (CAR) comprising an antigen-binding protein or fragment or derivative thereof, scFv, or fusion protein of any of embodiments 1-24.

32. The cell of embodiment 31, wherein the cell is a T cell or natural killer (NK) cell.

33. A method of inhibiting tumor growth or metastasis comprising contacting a tumor cell with an effective amount of an antigen-binding protein or fragment or derivative thereof, scFv, fusion protein, nucleic acid, expression vector, host cell, cell expressing a CAR, or pharmaceutical composition of any of embodiments 1-32.

34. A method of treating a neoplastic, hyperplastic, or hyperproliferative disorder in a subject in need thereof comprising administering a therapeutically effective amount of an antigen-binding protein or fragment or derivative thereof, scFv, fusion protein, nucleic acid, expression vector, host cell, cell expressing a CAR, or pharmaceutical composition of any of embodiments 1-32.

36. A method of treating cancer in a subject in need thereof comprising administering a therapeutically effective amount of an antigen-binding protein or fragment or derivative thereof, scFv, fusion protein, nucleic acid, expression vector, host cell, cell expressing a CAR, or pharmaceutical composition of any of embodiments 1-32.

36. A method of treatment comprising isolating T-cells from a subject, transfecting the T-cells with a vector comprising a nucleic acid encoding an isolated scFv comprising a VH and a VL linked by an amino acid spacer, wherein the VH and VL, respectively, comprise amino acid sequences selected from the group consisting of SEQ ID NOs: (i) 9 and 13; (ii) 9 and 15; (iii) 11 and 16; (iv) 9 and 17; (v) 12 and 14; (vi) 9 and 14; and (vii) 10 and 13, and administering the transfected T-cells to the subject.

37. The method of any of embodiments 34-36, wherein the subject has a cancer is selected from the group consisting of chronic myelocytic leukemia, multiple myeloma, acute lymphoblastic leukemia, acute myeloid/myelogenous leukemia, myelodysplastic syndrome, mesothelioma, ovarian cancer, gastrointestinal cancer, breast cancer, prostate cancer and glioblastoma.

38. The method of any of embodiments 33-37, further comprising administering a therapeutically effective amount of an effector cell and/or a cytokine.

39. The method of embodiment 38, wherein the cytokine is IL-15.

40. A kit comprising the antigen-binding protein or fragment or derivative thereof, scFv, fusion protein, nucleic acid, expression vector, host cell, cell expressing a CAR, or pharmaceutical composition of any of embodiments 1-32.

The present disclosure will be more readily understood by reference to the following example, which is provided by way of illustration and is not intended to be limiting.

### Example

### Materials and Methods

Cell and Cell Culture:

Human PBMC were isolated from whole blood by ficoll Hypaque density gradient separation. T cells were then isolated from PBMC by negative magnetic separation using magnetic beads containing antibodies against CD19, CD20, CD14, CD56 (Pan T-cell isolation kit, Miltenyi Biotech). Tap-deficient HLA-A2T2 cells, NK-92-MI-MI and all tumor cell lines were purchased from ATCC. Cells were cultured in RPMI 1640 with 2 mM L-glutamine and 10% Fetal Bovine Serum (FBS). NK-92-MI-MI cells and genetically CAR modified NK-92-MI-MI cells were propagated in Alpha Minimum Essential medium with 2 mM L-glutamine, 12.5% horse serum to a final concentration of 12.5% Horse Serum and 12.5% FBS.

MHC-Peptide Complexes:

All peptides were purchased and synthesized by Genscript Synthesis Inc. Biotinylated soluble MHC class I-peptide complexes were generated by refolding the peptides with recombinant HLA-A2 and β2 microglobulin at the Tetramer facility at MSKCC. In order to specifically biotinylate refolded monomeric MHC/peptide complexes, the heavy chain was expressed as a fusion protein containing a specific biotinylation site at the C-terminus. The PE conjugated MHC/peptide tetramers were obtained from the National Institutes of Health Tetramer Core Facility (Emory University, Atlanta, Ga.). The specific WT1 peptide used was RMFPNAPYL (WT1₁₂₆, SEQ ID NO: 1). The control peptides used included: (1) NLVPMVATV (SEQ ID NO: 28) derived from pp65 of human cytomegalovirus CMV, (2) RIITSTILV (SEQ ID NO: 29) abbreviated as Hud where RIITSTILV was derived from the protein HUD which was also called ELAVL4, (embryonic lethal, abnormal vision, drosophila]-like 4), (3) LLEEMFLTV (SEQ ID NO: 30) derived from CDR2 (cerebellar degeneration-related protein 2), (4) SLGEQQYSV (SEQ ID NO: 31) derived from WT1, (5) CMTWNQMNL (SEQ ID NO: 32) derived from WT1, (6) LMLGEFLKL (SEQ ID NO: 33) derived from Survivin, and (7) FLTPKKLQCV (SEQ ID NO: 34) derived from prostate specific antigen PSA.

Phage Display Selection:

The Tomlinson I+J human scFv phage display libraries (de Wildt, R. M., et al., Nat Biotechnol, 2000. 18(9): p. 989-94), containing approximately 2.85×10⁸ independent scFv clones, were used for selection according to previously published methods (Hu, J., et al., J Immunol, 2009. 183(9): p. 5748-55) with modifications. The 3.7×10¹² phages, from the combination of both libraries, were first preincubated with 50 μl of streptavidin paramagnetic Dynabeads (Dynal) and 20 μg unbiotinylated HLA-A2-NLVPMVATV (irrelevant complex) in 1 ml of PBS to deplete the streptavidin and HLA-A2 non-specific binders. The dynabeads were subsequently captured using a magnet and the supernatant (phage and irrelevant complex mixture) transferred to a separate tube containing 5 μg of biotinylated HLA-A2-RMFPNAPYL (WT1₁₂₆; SEQ ID NO: 1) and incubated at RT for 1 hour. The final mixture (1 ml) was then added to 100 μl of Dynabeads (preincubated with 2% milk and washed with PBS) and the contents were mixed for 30 min at RT with continuous rotation. The beads were then washed 10 times with PBS/0.1% Tween-20 and 3 times with PBS and the bound phage were eluted from the Dynabeads using 1 mg/ml trypsin in PBS (0.5 ml) for 20 min at RT. The phages were then used to infect TG1 E. coli (growing in log phase) at 37° C. in 20 ml of LB for 1 h. The 10¹² of M10KO7 helper phage was subsequently added to the mixture, further incubated for an additional 30 min, and the cells pelleted using centrifugation (3000 rpm for 10 min). The resulting cell pellet was resuspended in 200 ml LB containing Ampicillin (100 μg/ml) and Kanamycin (50 g/ml) and incubated overnight at 30° C. On the following morning, the overnight cultures were centrifuged at 3000 rpm for 15 min and the supernatant (180 ml) was mixed with polyethylene glycol (PEG8000)/NaCl solution on ice for 1 h so as to precipitate the amplified phage from the previous round of selection. The PEG/phage mixture was then centrifuged at 3000 rpm for 20 min, and some of the resulting phage pellet used for subsequent rounds of panning while the rest was frozen down in 15% glycerol at −80° C. Subsequent two rounds of panning were done using the same protocol as above with an increase in Dynabead washing steps and a decrease in the amount of biotinylated complexes used for selection.

After the final round of antibody selection, the eluted phages were used to infect both TG1 and HB2151 E. coli. TG1 cells were cultured overnight as mentioned above while the HB2151 cells were spread on 2YT plus Ampicillin (100 g/ml) agar plates. The next morning, individual colonies from the agar plate were picked and used to inoculate individual wells of a 48-well plate containing 400 μl LB plus Ampicillin (100 μg/ml)/well. After incubation for 3-6 hours at 37° C., 200 μl of 50% glycerol solution was added to each well and the plates stored at −80° C. as monoclonal stock cultures.

Mutagenesis by Error-Prone PCR:

Error-prone PCR of the entire scFv gene was performed using Stratagene GeneMorph® II Random Mutagenesis Kit according to the instructions of the manufacturer. Briefly, PCR was done in a 50 μL reaction containing 1×Mutazyme II reaction buffer, 0.5 μM each of primers ERROR Forward (5′ TCAGTTTTGGCCCAGGCGGCC 3′) (SEQ ID NO: 35) and ERROR Reverse (5′ ACCACTAGTTGGGCCGGCCTG 3′) (SEQ ID NO: 36), 0.2 mM (each) dNTPs, 1 ng of DNA template, 2 μM 8-oxo-deoxyguanosine triphosphate, 2 μM 2′-deoxy-p-nucleoside-5′-triphosphate, and 2.5 U of Mutazyme II DNA polymerase. The reaction mixtures were denatured at 95° C. for 2 min, cycled 35 times at 95° C. for 1 min, 60° C. for 1 min, and 72° C. for 1 min, and finally extended at 72° C. for 10 min. The PCR products were purified by 1% agarose gel electrophoresis and each amplified in four 100 μL PCR reactions containing 1×Accuprime PCR reaction mix (Invitrogen), 1 μM of primers YDRD Forward (5′-CTTCGCTGTTTTTCAATATTTTCTGTTATTGCTTCAGTTTTGGCCC-AGGCGGCC-3′) (SEQ ID NO: 37) and YDRD Reverse (5′-GAGCCGCCACCCTCAGAACCGCCACCCTC-AGAG-CCACCACTAGTTGGGCCGGCCTG-3′) (SEQ ID NO: 38), 120 ng of error-prone PCR product, and 2.5 U of Accuprime pfx DNA polymerase (Invitrogen). The reactions were thermally cycled at the same conditions except that 30 cycles were used. Reaction products were purified by 1% agarose gel electrophoresis and concentrated with ultrafilter in water.

Yeast Display Selection:

Construction and growth of yeast libraries were performed as previously described (Zhao, Q., et al., Mol Cancer Ther, 2011. 10(9): p. 1677-85). The selection for generating and isolating higher affinity mutants was as described in references (Zhao, Q., Z. Zhu, and D. S. Dimitrov, Methods Mol Biol, 2012. 899: p. 73-84) with some modifications. Briefly, induced yeast library (2×10⁹ cells) was subtracted by incubation with 10 μg-HLA-A2/ELMLGEFLKL (SEQ ID NO: 39)-conjugated magnetic beads for 1 h at RT in PBSA buffer (0.1% BSA in PBS), followed by separation with a magnetic stand. The subtracted yeast cells were subsequently incubated with 10 μg-HLA-A2/RMFPNAPYL (SEQ ID NO: 1)-conjugated magnetic beads for 3 h at RT in PBSA buffer. The magnetic isolated yeast cells were washed 3 times with PBSA buffer and added into 10 ml of SDCAA media for amplification overnight in a 30° C. shaker with 250 rpm. The amplified yeast cells were induced in SG/RCAA media for 18 h at 20° C. with 250 rpm shaking. During three fluorescence activated cell sorting (FACS) selections, yeast cells were respectively sorted at 100, 33 and 10 μg/ml biotinylated HLA-A2/RMFPNAPYL (SEQ ID NO:1). Sorting gates were determined to select only the population with higher antigen binding signals.

Expression and Purification of Soluble scFv and scFv-Fc:

The soluble scFv was expressed and purified as previously described Zhao, Q., et al., Mol Cancer Ther, 2011. 10(9): p. 1677-85; Zhao, Q., et al., Protein Expr Purif, 2009. 68(2): p. 190-5; Chen, W., et al., Mol Cancer Ther, 2012. 11(7): p. 1400-10). HB2151 cells were transformed with pComb3×plasmid containing scFv sequences. Single fresh colonies were inoculated into SB medium containing 100 μg/mL ampicillin and 0.2% glucose. The culture was grown at 37° C. with 250 rpm until OD₆₀₀ reached 0.5. Isopropyl-L-thio-h-D-galactopyranoside (final concentration 0.5 mM) was added to induce expression. After overnight growth at 30° C., the bacteria were centrifuged at 5,000×g for 15 min. The pellet was resuspended in PBS with polymyxin B (10,000 units/mL). Soluble scFv was released from periplasm by incubating at 30° C. for 30 min. The extract was clarified at 15,000×g for 30 min. The clear supernatant was recovered for the purification on Ni-NTA column.

The scFv-Fc variant genes were synthesized for CHO cells (Genscript). Using the bluescript vector, these scFv-Fc genes were transfected into CHO-s cells and selected with G418 (Invitrogen) as previously described (Cheung, N. K., et al., Oncolmmunology, 2012. 1(4): p. 477-486). The scFv-Fc producer lines were cultured in Opticho serum free medium (Invitrogen), and the mature supernatant was harvested as previously described (Tassev, D. V., M. Cheng, and N. K. Cheung, Cancer Gene Ther, 2012. 19(2): p. 84-100). The soluble scFv-Fc protein was purified using the MabSelect affinity chromatograph medium (GE Healthcare). The affinity column was pre-equilibrated with 25 mM sodium citrate buffer with 0.15 M NaCl, pH 8.2. Bound protein was eluted with 0.1 M citric acid/sodium citrate buffer, pH 3.9 and alkalinized (1:10 v/v ratio) in 25 mM sodium citrate, pH 8.5. The eluted scFv-Fc was subsequently concentrated using a 50,000 MWCO Vivaspin centrifuge tube (Sartorius Stedim) and tested for its ability to bind to recombinant antigen using ELISA as well as natively presented peptide on the surface of T2 cells using flow cytometry.

ELISA Assay:

The specificity of individual phage clones, soluble scFv and scFv-Fc antibodies was assessed by ELISA at RT with indirectly coated HLA-A2/peptide complexes. Vinyl flat bottom microtiter plates (Thermo Fisher) were used for ELISA assays. Plates were initially coated overnight at 4° C. with BSA-biotin (10 g/ml; 50 μl/well). The next day, the contents were discarded and the plates incubated at RT with streptavidin (10 g/ml; 50 μl/well) for 1 h. The contents were discarded and the plates incubated with recombinant biotinylated HLA-A2/peptide complexes (5 μg/ml; 50 μl/well) at RT for 1 h. The plates were then incubated with 2% milk PBS (150 μl/well) at RT for 1 h. After blocking, the plates were washed 3 times with PBS and then incubated with bacterial supernatant from their respective HB2151 culture plate wells, purified scFv, or purified scFv-Fc at RT for 1 h. After the contents were discarded, the plates were washed 5 times with PBS, and then incubated with a horse radish peroxidase (HRP) conjugated mouse-anti-Flag tag antibody (1:5000 dilution, Sigma Aldrich) to detect the scFv, or a HRP conjugated goat-anti-human Fc antibody (0.5 μg/ml, Jackson Immunoresearch Laboratories) to detect the scFv-Fc. The plates were developed using o-phenylenediamine (OPD) buffer (150 μl/well), which was made by combining 20 mg of OPD tablets in 40 ml of citrate phosphate buffer with 40 μl 30% hydrogen peroxide. The color reaction was stopped by adding 30 μl of 5N sulfuric acid to each well, and the plates read using the Dynex MRX ELISA plate reader at 490 nm. Lastly, the contents of the scFv plates were discarded, the plates washed 5 times with PBS, and developed according to the method above.

Surface Plasmon Resonance:

Kinetics and affinities of various antibodies and WT1₁₂₆/HLA-A2 were analyzed by surface plasmon resonance technology using a Biacore T100 instrument (GE healthcare). Biotinylated WT1₁₂₆/HLA-A2 was captured by streptavidin-fusion protein on a sensor chip (CM5). A control reference surface was prepared for nonspecific binding and refractive index changes. For analysis of the kinetics of interactions, varying concentrations of antibodies were injected at flow rate of 30 μl/min using a running buffer containing 10 mM HEPES, 150 mM NaCl, 3 mM EDTA, and 0.05% Surfactant P-20 (pH 7.4). The association and dissociation phase data were fitted simultaneously to a 1:1 model by using BIAevaluation 3.2. All the experiments were done at 25° C.

Flow Cytometric Analysis:

For the staining of T2 cells presenting HLA-A2/peptide, T2 cells were harvested and transferred to serum-free IMDM containing 20-25 μg/ml β2-microglobulin (β₂M). The T2 cells were then incubated with 4 μM or less of either WT1₁₂₆ peptide or any number of control peptides, at 37° C. for 5 hours. Cells were then incubated with specific purified scFv-Fc for 30 min on ice, washed, and incubated with secondary antibody reagents when necessary. A similar method was used for epitope mapping experiments, except that T2 cells were incubated with either wild-type or alanine substituted WT1₁₂₆ peptides at 37° C. overnight. Analysis was performed using a BD Bioscience FACScalibur. The same method was used to determine the binding of the antibodies to tumor cells and cell lines. For the staining of CAR-expressing cells, all fluorescent antibodies for surface staining were purchased from BD Biosciences. The CAR expression was analyzed on human T cells using anti-CD4, anti-CD8, and MHC/peptide tetramers, and on NK-92-MI, PG-13 and K562 cells using MHC/peptide tetramers and the reporter GFP.

Generation of HLA-A2/WT1₁₂₆-Specific CAR Construct:

The protocol of CAR was obtained from Dr. Dario Campana at St. Jude Children's Hospital and previously described (Imai, C., et al., Leukemia, 2004. 18(4): p. 676-84). The scFv sequences were fused in-frame to the scFv-4-1BB-CD3 DNA constructs that were synthesized by Genscript. The CAR gene was under a CMV promoter which was followed by IRES-GFP. The entire CAR genes were subsequently excised and inserted into the expression vector. The ligation products were then transformed into E. coli, plated on LB plus Ampicillin (100 g/ml). Once the sequences were validated, the DNA was packaged into retroviruses and used to infect human T cells, K562 or NK-92-MI cells.

Retroviral Production and Transduction:

For T-cell or K562 transduction, vector DNA was transfected into H29 packaging cells in the presence of CaCl₂. Viral supernatant was collected for two consecutive days and stored. The packaging cell line PG-13 was then transfected with viral supernatant generated using H29 cells. PG-13 cells expressing the transduced vector DNA were sorted using GFP as the selection marker, cloned and expanded, and culture supernatants were collected for T-cell transduction. Purified T-cells were then stimulated with CD3/CD28 beads for 24 hours. PG-13 viral supernatant was added to retronectin coated plates, T-cells were then added, and the plates were spun down and incubated for 48 hours. T-cells or K562 expressing the transduced vector were detected using GFP as well as WT1₁₂₆/HLA-A2 tetramer by FACS.

For transduction of NK-92-MI cells, the following procedure was employed which used a 293T-based retroviral production cell line (GP2). Briefly, 7 μg of CAR DNA was combined with 3.5 μg of PCLAmpho helper construct and 3.5 μg pVSVg in 1 ml of serum-free DMEM. This mixture was then combined with 1 ml serum-free DMEM containing 36 μl of Lipofectamine 2000 (Invitrogen) and incubated at RT for 20 min. Afterwards, the DNA-Lipofectamine complex (2 ml) was mixed with GP2 cells (3-5×10⁶) in 10 ml of DMEM containing 10% FBS and cultured at 37° C. for 72 h. Subsequently, the supernatant (12 ml) was depleted of GP2 cells during recovery and incubated with 3 ml Lenti-X Concentrator solution (Clontech) at 4° C. for 12-16 h. Afterwards, the solution was centrifuged at 3000 rpm for 15 min, the supernatant discarded, and the pellet dissolved in 1 ml complete Alpha Essential medium. NK-92-MI cells were then incubated for 72 h and analyzed by flow cytometry for CAR expression using GFP and an WT1₁₂₆/HLA-A2-PE(phycoerythrin)-labeled tetramer.

Cytotoxicity Assay:

Antibody-dependent cell-mediated cytotoxicity (ADCC) assays were performed using NK-92-MI cells stably transfected with the human CD16 Fc receptor as previously described (Cheung, N. K., et al., Oncolmmunology, 2012. 1(4): p. 477-486). Target tumor cells were collected with 2 mM EDTA in Ca/Mg free PBS and washed in RPMI medium, before radiolabeling with ⁵¹Chromium for ADCC assays. The percentage of ⁵¹Cr Release was determined using the following formula: ((Sample Release−Spontaneous Release)/(Total Release−Spontaneous Release))×100.

A standard ⁵¹Cr release assay evaluated in-vitro T-cell or NK-92-MI mediated cytotoxicity as previously described (Tassev, D. V., M. Cheng, and N. K. Cheung, Cancer Gene Ther, 2012. 19(2): p. 84-100; Koehne, G., et al., Blood, 2002. 99(5): p. 1730-40; Koehne, G., et al., Blood, 2000. 96(1): p. 109-17). The cytolytic capacity of T-cells or NK-92-MI cells was also tested against HLA-A2/WT1⁺ tumor cell lines as well as autologous EBV-BLCL loaded with the WT1₁₂₆ peptide. Alloreactivity was assessed using HLA mismatched EBV-BLCL, and NK like activity was evaluated against the erythroleukemia cell line K562 lacking the expression of HLA but with high expression of WT1.

Molecular Modeling:

Molecular modeling, energy calculations, docking simulations, and image renderings were done using Discovery Studio 4.0 (Accelrys, San Diego, Calif.) or Pymol (Schrodinger LLC, New York, N.Y.). A homology model of the anti-WT1-HLA-A2 scFv antibody was built using pdb structure of the anti-SARS scFV antibody from pdb 2GHW as a template (68% sequence identity). Each CDR loop was then refined using additional homologous templates shown in parentheses: L1 (2BX5, 1RZI, 2UZI), L2 (2VH5,2UZI, 2BX5), L3 (2BX5, 3NCJ, 2FGW), H1 (2QQN, 1H3P, 3QOS), H2 (2QQN, 3SKJ, 3SOB), and H3 (1MRD, 1MRE, 1MRC). The final model underwent a 2 ns molecular dynamics simulation to reach a low energy conformation for use in docking simulations. Docking simulations were run using ZDOCK using the energy minimized homology model of anti-WT1-HLA-A2 scFv with the crystal structure of HLA-A2-WT1-RMF (pdb 3HPJ).

Therapy of Human Leukemia Xenograft Models:

Two million BV173 human leukemia cells were injected intravenously into Rag2(−/−)gammaC(−/−) double knockout (DKO) mice. On day 6, tumor engraftment was confirmed by briefly luciferase imaging in all mice that were to be treated. Mice were randomly assigned to treatment and control groups (n=5). Antibodies (50 μg/mouse) were administered intravenously twice a week for a total of 4 doses. In animals that also received human effector cells with or without antibodies, PBMCs from healthy donor (10 million cells per i.v. injection) and cytokine IL15/IL15α complex (10 μg per s.c. injection) were injected intravenously into mice (10 million cells per mouse) on day 7 and 14. Tumor growth was assessed by luminescence imaging once to once a week.

### Results

Selecting for Human scFvs Specific for HLA-A2/WT1₁₂₆ Using Phage Display:

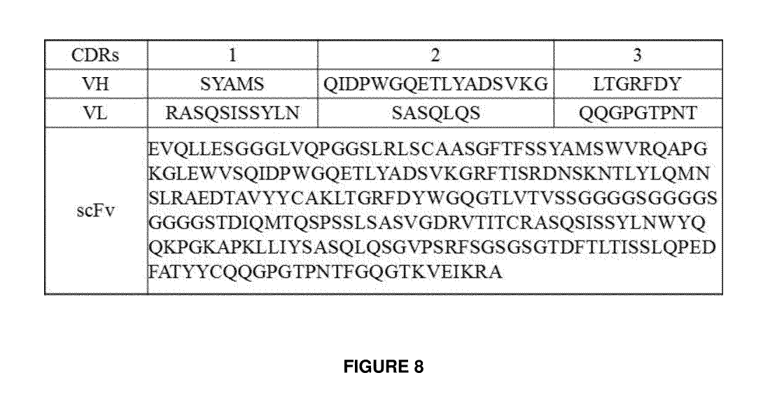

The phage display approach was used to select for TCR-like antibodies. With the assumption that TCR-like antibodies are under-represented in a mature B-cell library (Dahan, R. and Y. Reiter, Expert Rev Mol Med, 2012. 14: p. e6), the recombinant “Tomlinson I+J” human scFv library was chosen. In order to eliminate phages which cross-react with the framework of HLA-A2 or the streptavidin beads themselves, clone selection was first performed using a negative screen on HLA-A2/pp65 control peptide monomers before the positive screen on HLA-A2/WT1₁₂₆ monomers. Finally, phage binders cross-reactive with irrelevant recombinant HLA-A2/peptide complexes were discarded. From 48 clones, three individual scFv clones were isolated that bound specifically to the HLA-A2/WT1₁₂₆ complex. Further analysis revealed that all three clones had the exact same DNA sequence and this clone was designated as Clone45 (FIG. 8). After expression and purification, Clone45 scFv was retested against a larger panel of HLA-A2/peptide complexes. As was seen in the initial ELISA screen, Clone45 scFv maintained its specificity towards its targeted HLA-A2/WT1₁₂₆ and did not show binding to other irrelevant HLA-A2/peptide complexes or to the WT1₁₂₆ peptide itself. The TCR-like binding of Clone45 was confirmed by flow cytometry of T2 cells loaded with WT1₁₂₆ peptide, but not to T2 cells loaded with control peptide pp65 (data not shown).

Selection of Higher-Affinity scFv Mutants Using the Yeast Display:

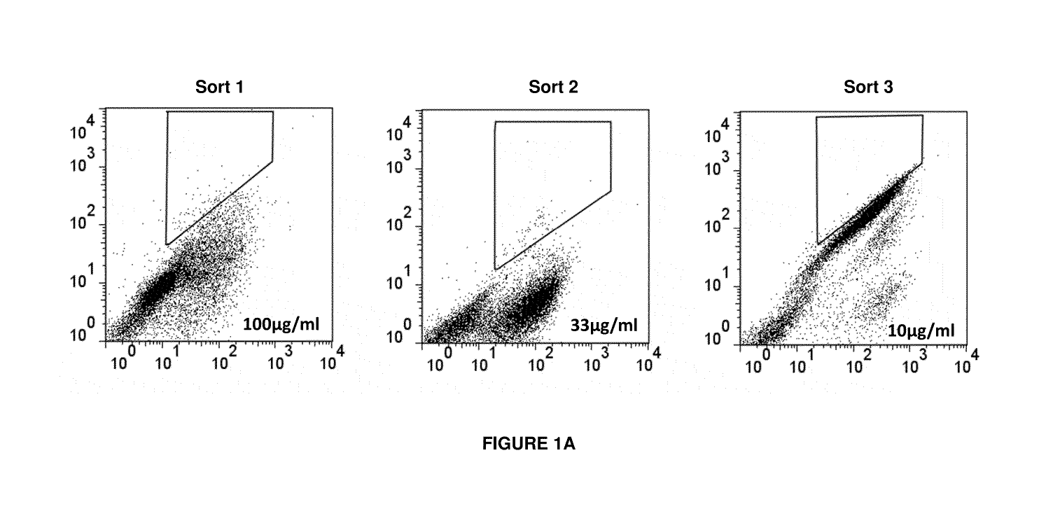

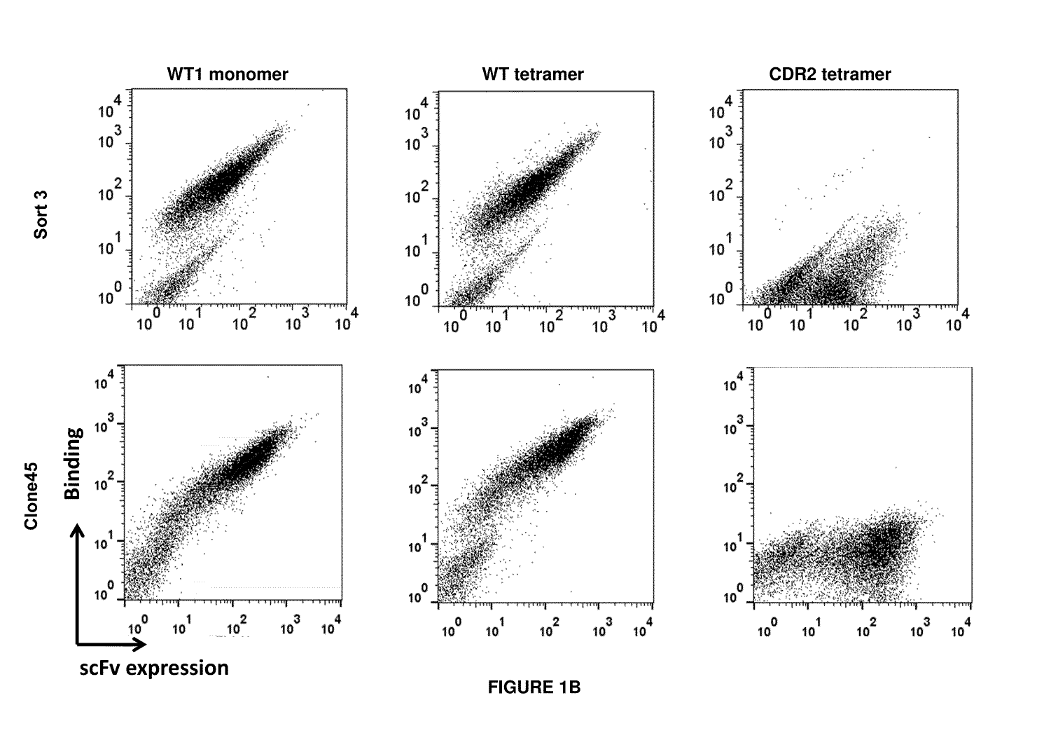

By Biacore, the binding affinity of scFv Clone45 was low (K_{D}=300 nM) (Table 2), compared to therapeutic antibodies commonly used in clinic (Wittrup, K. D., et al., Methods Enzymol, 2012. 503: p. 255-68; Dimitrov, D. S. and J. D. Marks, Methods Mol Biol, 2009. 525: p. 1-27, xiii). To affinity mature Clone45, randomly diversified libraries were created, comprised of scFv mutants with low (<5/1000 bp), moderate (5-9/1000 bp), and high (>9/1000 bp) mutation rates, displayed on yeast cells by homologous recombination with a vector containing a C-terminal Aga2 protein and c-myc tag (Zhao, Q., et al., Mol Cancer Ther, 2011. 10(9): p. 1677-85). The final antibody library contained 5×10⁸ independent clones and was subjected first to one round of selection by using the HLA-A2/WT1₁₂₆ conjugated magnetic beads. This allowed elimination of yeast cells that did not express antibodies or bound weakly to HLA-A2/WT1₁₂₆. Sequential FACS sorting was carried out 3 times with stringent mean fluorescence intensity (MFI) for binding to HLA-A2/WT1₁₂₆ (FIG. 1A). To demonstrate specificity, the final sorted yeast mutants were shown to stain positively with either the monomer or tetramer of HLA-A2-WT1₁₂₆ peptide, but not with an irrelevant HLA-A2/CDR2-derived peptide (LLEEMFLTV) (SEQ ID NO: 30) (FIG. 1B). The highest affinity clones from the final round of sorting were sequenced.

TABLE 2
Binding rate constants and affinities of scFvs or scFv-Fc by Biacore.

| Antibodies | kₒₙ (M⁻¹s⁻¹) | k_{off} (s⁻¹) | K_{D}(nM) |
|---|---|---|---|
| Clone45 scFv | 273000 | 0.0718 | 263 |
| S3.1 scFv | 148000 | 0.00191 | 12.9 |
| S3.3 scFv | 22000 | 0.0000535 | 2.43 |
| S3.6 scFv | 125500 | 0.00177 | 14.1 |
| Q1L scFv | 94300 | 0.00550 | 58.3 |
| Q2L scFv | 115000 | 0.000355 | 3.08 |
| Q2L scFv-Fc | 484000 | 0.00000115 | 0.002 |

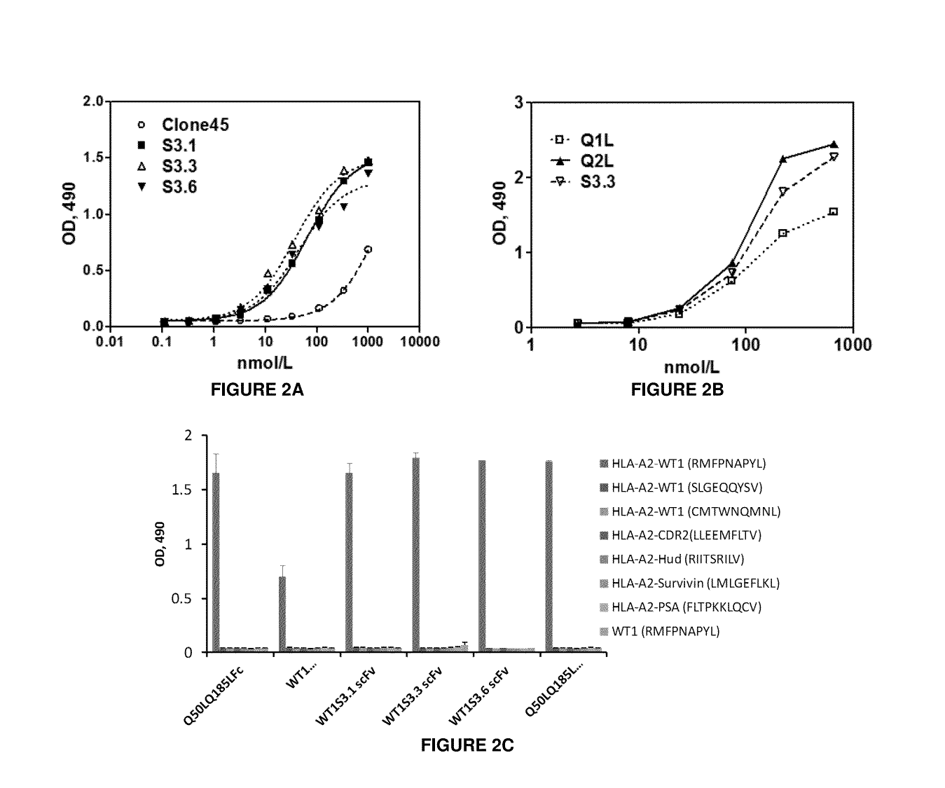

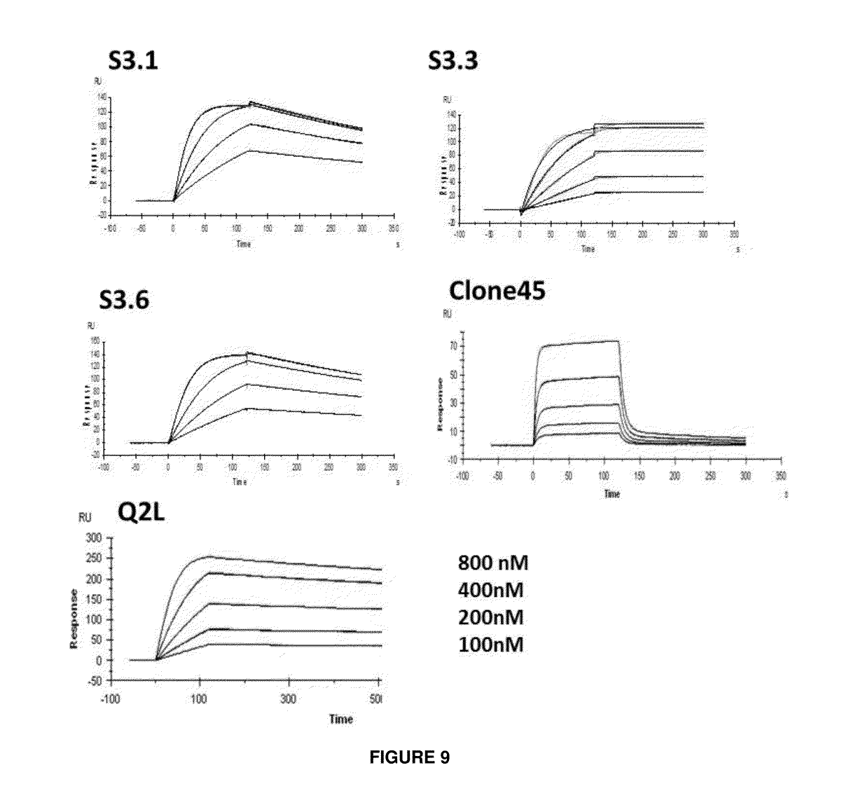

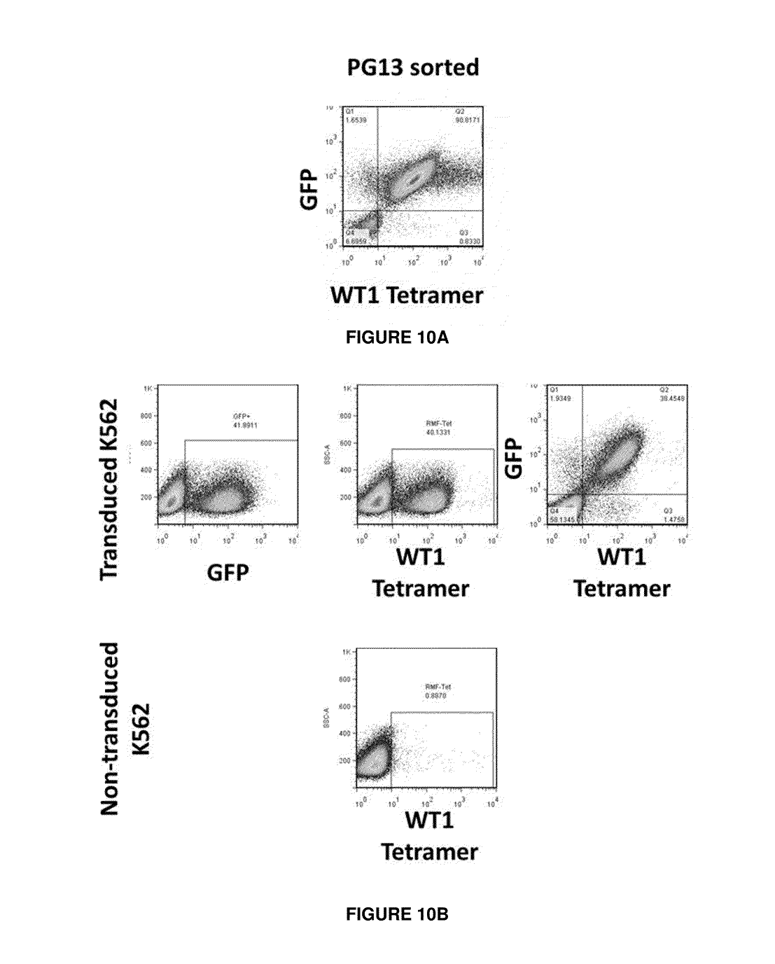

When the sequence data were grouped by cluster analysis, 4 repeatedly selected scFv sequences (S3.1, S3.3, S3.6 and S3.22) were identified. Compared with parental clone Clone45, the most dominant mutations contained 9 amino acid substitutions in the variable regions of the heavy chain and light chains (Table 3). All three scFv (S3.1, S3.3 and S3.6) exhibited a stronger binding signal than parental scFv Clone45 at all concentrations by ELISA on HLA-A2/WT1₁₂₆ complex (FIG. 2A). All three scFvs maintained the binding specificity of scFv Clone45 and did not cross-react with other HLA-A2 complexes displaying irrelevant peptides (FIG. 2C). As shown in Table 2, the three scFvs (S3.1, S3.3 and S3.6) bound HLA-A2/WT1₁₂₆ monomer with dissociation constants (K_{D}) of 13 nM, 2.5 nM and 14 nM, respectively, compared to K_{D}=263 nmol/L of parental Clone45. With a K_{D} of 2.4 nM, scFv S3.3 exhibited the highest improvement in binding affinity of nearly 100-fold, with a significantly prolonged dissociation time (FIG. 9).

TABLE 3
Listing of 4 most frequent sequences selected
from the yeast display library

| Amino | Heavy | | | Light | | | | | | Frequency |
|---|---|---|---|---|---|---|---|---|---|---|
| acid | chain | | | chain | | | | | | of |
| position | 28 | 50 | 87 | 1 | 30 | 53 | 65 | 70 | 77 | clones |
| Clone45 | T | Q | R | D | S | Q | S | D | S | |
| S3.1 | T | L | G | D | S | Q | S | D | N | n = 11 |
| S3.3 | T | L | R | N | S | L | N | D | S | n = 14 |
| S3.6 | T | L | R | D | N | Q | S | V | S | n = 1 |
| S3.22 | L | L | R | D | S | Q | S | D | S | n = 3 |

The mutated residues are underlined. Amino acid positions are given as Kabat numbers (hoot://www.imgt.org.).

Identifying Crucial Amino Acid Positions for Affinity Maturation of TCR-Like Antibodies:

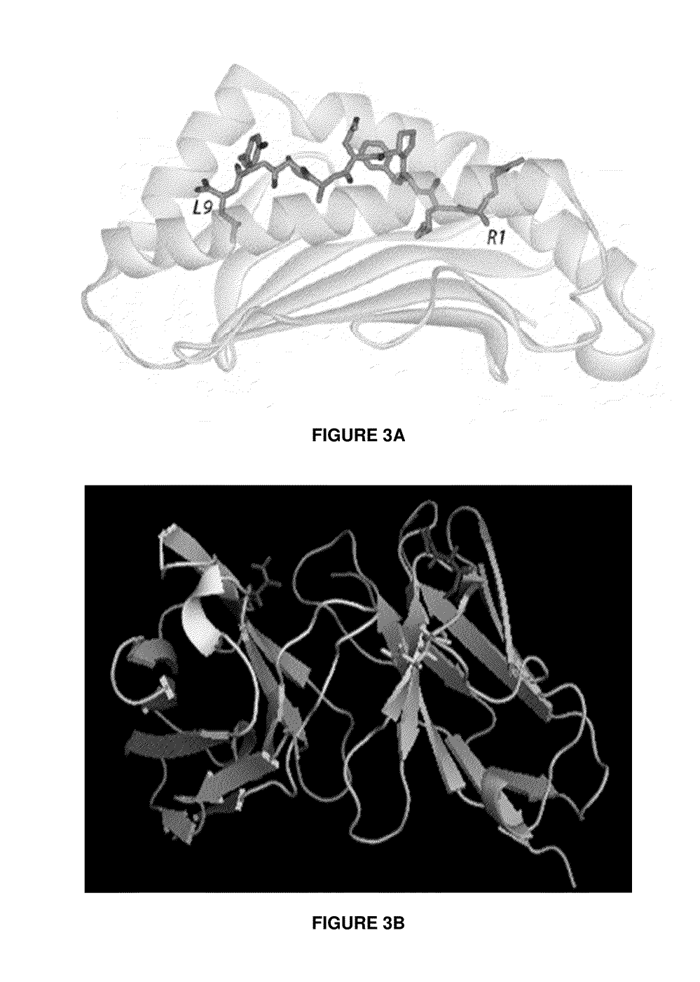

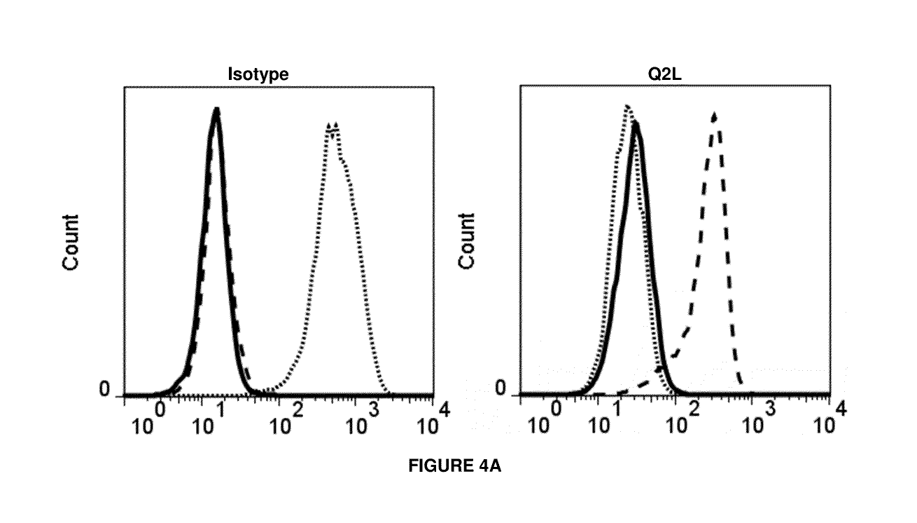

For affinity maturation the identification of key residues as the interaction of antibody and its antigen was crucial Stewart-Jones, G., et al., Proc Natl Acad Sci USA, 2009. 106(14): p. 5784-8; Li, Y., et al., Nat Struct Biol, 2003. 10(6): p. 482-8). The crystal structure of WT1₁₂₆ bound to HLA-A2 at 2 Å resolution has revealed the usual architecture of class I MHC/peptide complexes Borbulevych, O. Y., et al., Mol Immunol, 2010. 47(15): p. 2519-24). TCR-like antibodies are known to recognize MHC-bound peptides either by contacting the peptide directly, as a TCR usually does, or by recognizing a unique conformation of the MHC protein bound to a particular peptide (Mareeva, T., E. Martinez-Hackert, and Y. Sykulev, J Biol Chem, 2008. 283(43): p. 29053-9). TCR generally recognizes the extended conformation characterized by a bulge at Proline (P) and Asparagine (N) at residues 4 and 5 of the WT1₁₂₆ peptide (FIG. 3A). The structure of the scFv Clone45 was generated using homology modeling (FIG. 3B). The CHARMm force field was then used to perform energy minimizations and molecular dynamic simulations of the structure. The alignment of 4 scFv mutants (Table 3) suggest Q50L in the heavy chain as the first critical position for affinity maturation. Q53L in the light chain of the best mutant (S3.3) was the second position. Based on homology modeling, these two glutamine residues located in CDR2 regions of heavy and light chains, respectively, were involved in antigen recognition (FIG. 3B).

Binding Properties and Specificity of TCR-Like Antibodies:

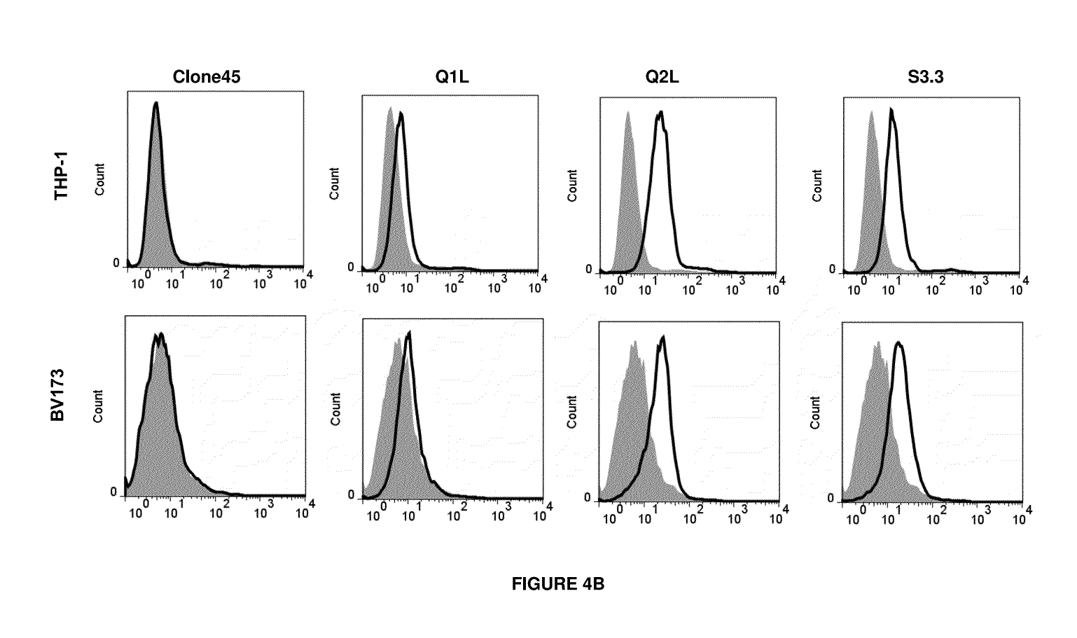

To confirm the predicted “hotspots”, scFv Q1L with VH-Q₅₀L mutation and scFv Q2L with VH-Q₅₀L/VL-Q₅₃L mutations were created. The scFv Q1L contained only one amino acid change in the heavy chain, whereas scFv Q2L had an additional light-chain mutation. ScFv Q2L exhibited an equivalent binding signal to S3.3 (the scFv mutant with the highest affinity) in a dose-responsive fashion by ELISA on HLA-A2-WT1₁₂₆ complexes, whereas scFv Q1L showed weaker binding than S3.3 (FIG. 2B). By Biacore, Q2L showed comparable affinity (K_{D}=3 nM) to S3.3 (K_{D}=2.4 nM) while Q1L was lower (K_{D}=58 nM) (Table 3). When reshaped into a scFv-Fc fusion protein, Q2L showed an even higher apparent affinity (2 pM). By ELISA (FIG. 2C), affinity-maturated Q2L as scFv or scFv-Fc, maintained its specificity towards its targeted HLA-A2/WT1₁₂₆ with no cross-reactivity with other HLA-A2-peptide complexes, including WT1 187-195 (SLGEQQYSV) (SEQ ID NO: 31) and 235-243 (CMTWNQMNL) (SEQ ID NO: 32), or with the WT1 126-134 (RMFPNAPYL) (SEQ ID NO: 1) peptide by itself. Specificity was further confirmed by the binding of Q2L to T2 cells pulsed with analog WT1₁₂₆ peptides (FIG. 4A), and no binding to T2 cells alone or T2 cells pulsed with irrelevant HLA-A2 binding peptides.

Affinity-maturated antibodies were able to recognize the naturally processed WT1 epitope presented by HLA-A2 molecules on the cell surface in a panel of tumor cell lines (Table 4). Q2L showed positive staining of human tumor cell lines positive for both HLA-A2 and WT1, but not to cell lines that were either HLA-A2(−) or WT1(−). The intensity of binding was correlated with expression level of HLA-A2 molecule. Cell lines that were genotypically positive for HLA-A2 with little HLA-A2 expression were also negative for binding to Q2L. The binding of Clone 45 (low affinity), Q1L (modest affinity), Q2L or S3.3 (high affinity) against WT1/HLA-A2 positive leukemia cell lines was compared. As expected, MFI correlated with antibody affinities (FIG. 4B). These results confirmed that two crucial leucine mutations at glutamine residues of CDRs afforded the pM affinity maturation of scFv-Fc against HLA-A2/WT1₁₂₆.

TABLE 4
Expression of HLA-A2 and immunostaining of Q2L scFv-Fc on tumor cell
lines.

| Cell line | Tumor type | Ratio (BB7.2/isotype)* | Ratio (Q2L/isotype)* |
|---|---|---|---|
| K562 | Leukemia | 1.1 | 1.1 |
| K562-HLA-A2 | Leukemia | 28.8 | 2.5 |
| Molt-4 | Leukemia | 1.1 | 1.0 |
| THP-1 | Leukemia | 53.9 | 35.6 |
| BA25-17 | Leukemia | 118.5 | 7.2 |
| BA25-69 | Leukemia | 113.6 | 19.1 |
| BV-173 | Leukemia | 209.2 | 20.0 |
| SKN-JC-1 | NB | 86.8 | 5.1 |
| SKN-JC-2 | NB | 53.3 | 3.5 |
| SKNJB | NB | 6.4 | 3.2 |
| LAN-1 | NB | 4.5 | 6.2 |
| SKNLD | NB | 0.9 | 1.0 |
| SKMEL-5 | Melanoma | 32.1 | 9.0 |
| JMN | Mesothelioma | 165.3 | 10.2 |
| U87 | Glioblastoma | 77.5 | 10.8 |
| U251 | Glioblastoma | 16.2 | 3.2 |
| U2 OS | Osteosarcoma | 21.7 | 2.1 |
| MDA-MB-231(HTB-26) | Breast | 110.9 | 6.5 |
| MDA-MB-361(HTB-27) | Breast | 0.9 | 1.4 |
| MDA-MB-468(HTB- | Breast | 1.1 | 1.1 |
| 132) | | | |
| SKBR3 | Breast | 1.1 | 1.1 |
| MCF-7 | Breast | 9.9 | 2.1 |
| SKOV-3 | Ovarian | 1.0 | 1.2 |
| OVCAR-3 | Ovarian | 7.8 | 1.2 |
| OVCAR3-pp65 | Ovarian | 10.0 | 1.4 |
| Colo 205 | Colon | 32.6 | 2.2 |
| Caco-2 (HTB-37) | Colon | 26.8 | 2.9 |
| HTB37-pp65/GFP | Colon | 10.9 | 2.3 |
| SW480 | Colon | 40.5 | 3.2 |
| SKHEP-1 | Liver | 28.3 | 7.5 |
| HepG2 | Liver | 14.4 | 3.4 |
| NCI-H345 | Small cell lung | 6.2 | 1.7 |
| | cancer | | |
| NCI-H522 | Non-small cell lung | 15.8 | 1.5 |
| | cancer | | |
| SK-ES-1 | Ewing's sarcoma | 7.7 | 1.4 |
| JN-DSRCT | Desmoplastic small | 5.1 | 1.3 |
| | round cell tumor | | |

* ratio of mean of fluorescence intensity

Epitope Mapping:

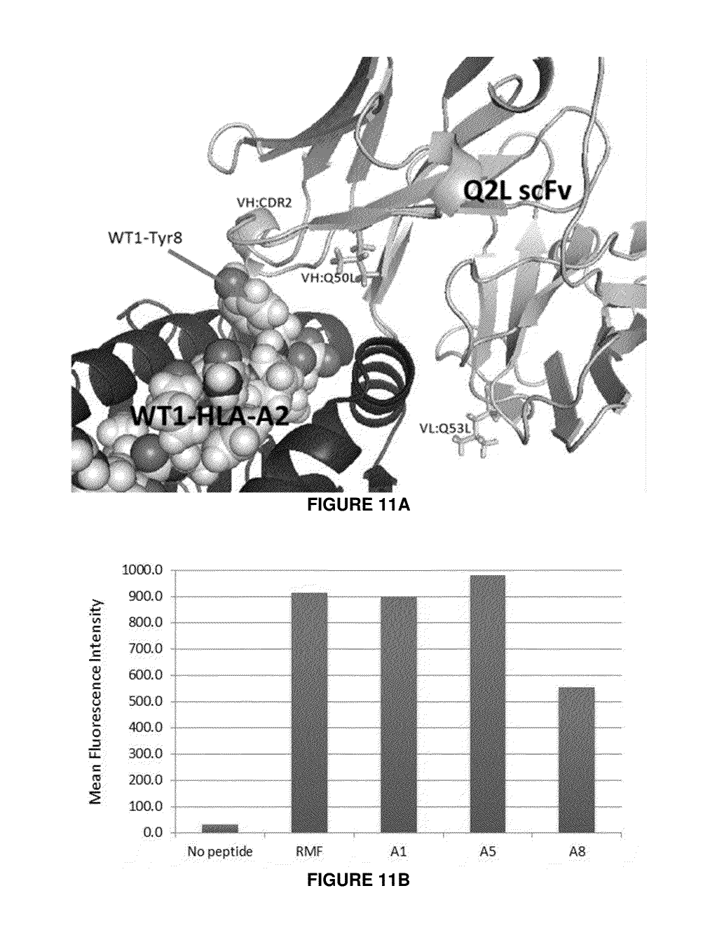

To confirm the precise molecular epitope of the Q2L scFv, both in silico docking simulations and experimental binding with alanine-substituted WT1₁₂₆ peptides were used (FIG. 11A and FIG. 11B). For in silico modeling, a homology model of Q2L scFv was docked onto the known crystal structure of HLA-HLA-A2/WT1₁₂₆. The top docked pose (FIG. 11A) revealed that the binding epitope involves the interaction of the heavy chain CDR2 of the Q2L scFv with Tyr 8 of WT1₁₂₆. The mutation VH-Q₅₀L enhances this interaction at this site. The model shows that the second mutation VL-Q₅₃L enhances the interaction of Q2L with the helical peptide-binding cleft of the HLA molecule. The predicted epitope was verified with binding experiments using WT1₁₂₆ peptides substituted with alanine at positions 1, 5, and 8 (FIG. 11B). T2 cells were pulsed with these peptides, and Q2L binding was measured by flow cytometry. Reduced binding was only observed when Tyr8 was mutated to Ala, confirming the epitope.

Antibody-Dependent Cell-Mediated Cytotoxicity (ADCC):

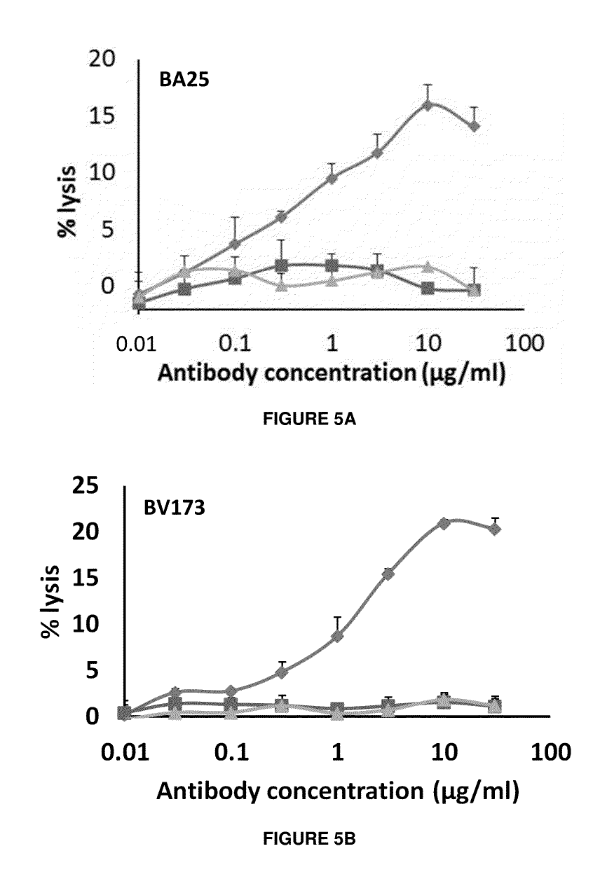

The ability of Q2L scFv-Fc to induce mediate ADCC of leukemia targets carrying the HLA-A2/WT1₁₂₆ complex was tested. For ADCC, NK-92-MI cells transfected with human CD16 were used (Cheung, N. K., et al., Oncolmmunology, 2012. 1(4): p. 477-486). Q2L mediated dose-dependent ADCC against the WT1₁₂₆ epitope naturally presented by HLA-A2 molecules on BV173 and BA25 leukemia targets (FIG. 5A and FIG. 5B). The parental Clone45 and the irrelevant isotype matched TCR-like scFv-Fc antibody (HLA-A2/Hud) did not kill these tumor cells. Complement-mediated cytotoxicity (CMC) was ineffective (data not shown).

Arming NK Cells and T Cells with Chimeric Antigen Receptor (CAR):

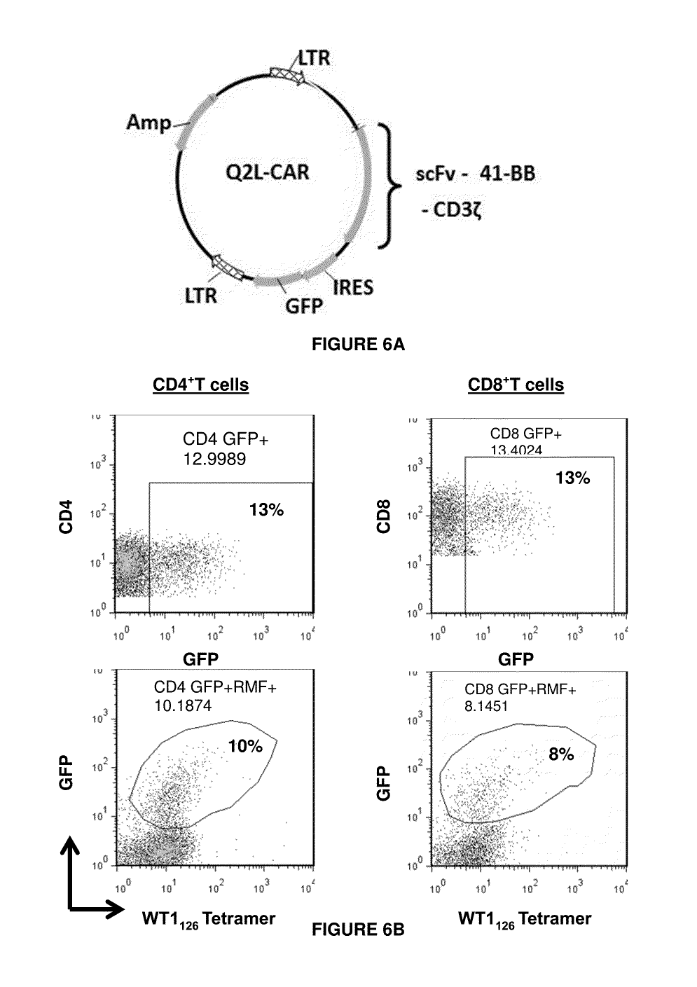

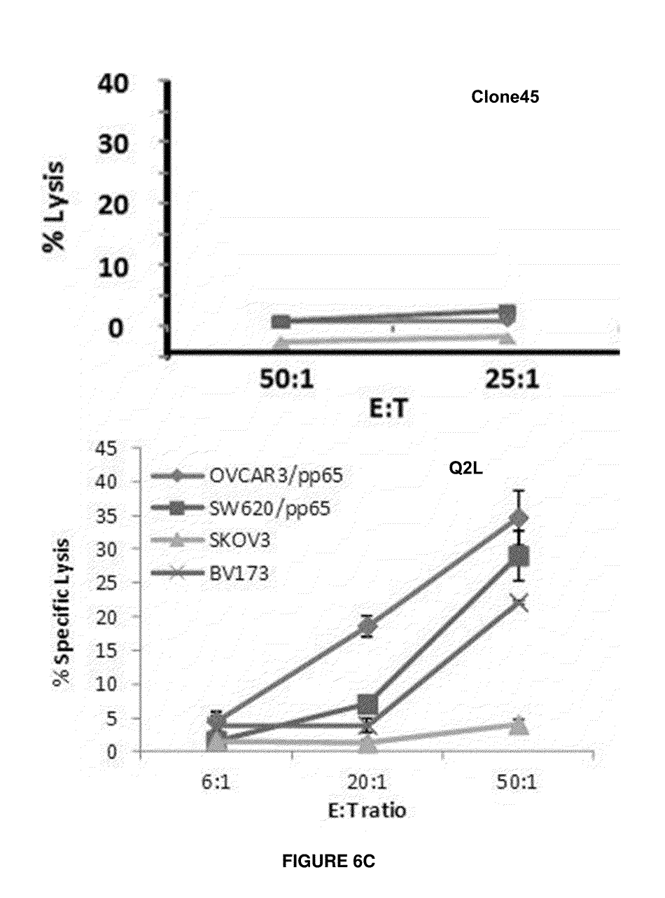

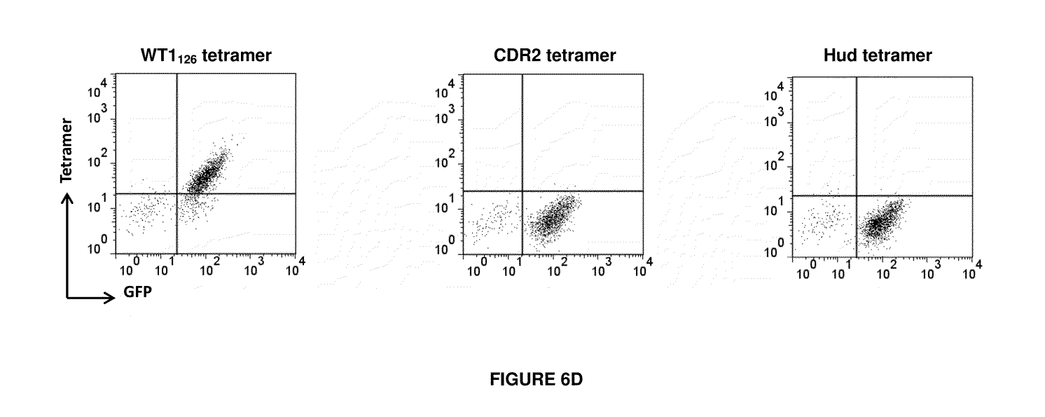

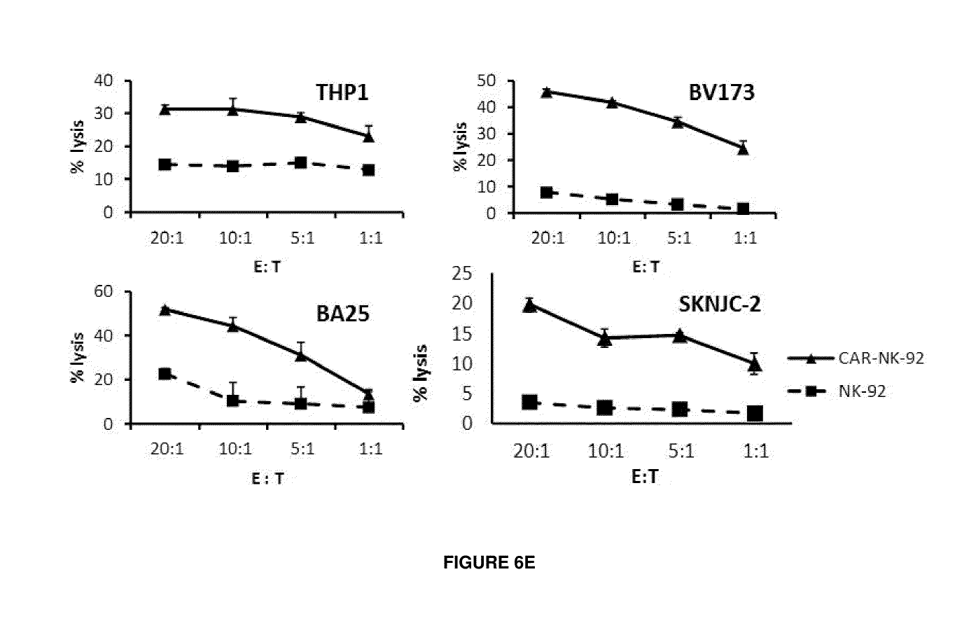

CAR was constructed using the Q2L scFv linked to the intracellular signaling domains of 4-1BB and CD3 (FIG. 6A). NK-92-MI cells were genetically modified to express Q2L CAR using retroviral MSCV vector carrying a IRES-GFP sequence downstream used for FACS sorting, in order to produce a fairly pure population (˜90%) of stable NK-92-MI cells carrying anti-HLA-A2/WT1₁₂₆ CAR on their cell surface (FIG. 6D). Their antigen specificity was confirmed by specific tetramer staining. When tested against HLA-A2(+) and WT1(+) leukemia cell lines (THP-1, BV173 and BA25) or neuroblastoma cell line (SKNJC2) specific lysis was observed only with NK-92-MI-scFv(Q2L), but not with unmodified NK-92-MI cells (FIG. 6E).

CD3(+) T cells isolated from the peripheral blood of healthy donors, using retroviral transduction in vitro were modified with either the Q2L-CAR or the Clone45-CAR. Transduction efficacy varied between 20% and 40%, and correct functional assembly of immune receptors was confirmed by HLA-A2/WT1₁₂₆ tetramer staining (FIG. 6B). Low affinity Clone45-CAR did not stain well with the tetramer and the CAR-modified T cells were not cytotoxic for WT1(+) HLA-A2(+) tumor targets (data not shown). In contrast, the high affinity Q2L-CAR bound strongly to the tetramer and mediated efficient tumor lysis in a dose-dependent manner (FIG. 6C). Q2L-CAR grafted T cells specifically recognized and killed HLA-A2(+)/WT1(+) targets (e.g. BV173, SW620/pp65, OVCAR3/pp65 in a dose dependent manner, but not HLA-A2(+)/WT1(−) cells (SKOV3).

Therapy of Human Leukemia Cells by Q2L In Vivo:

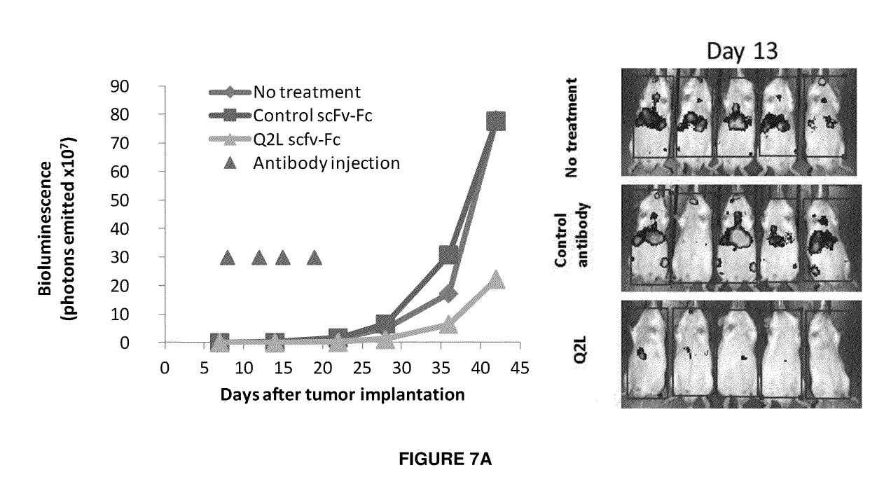

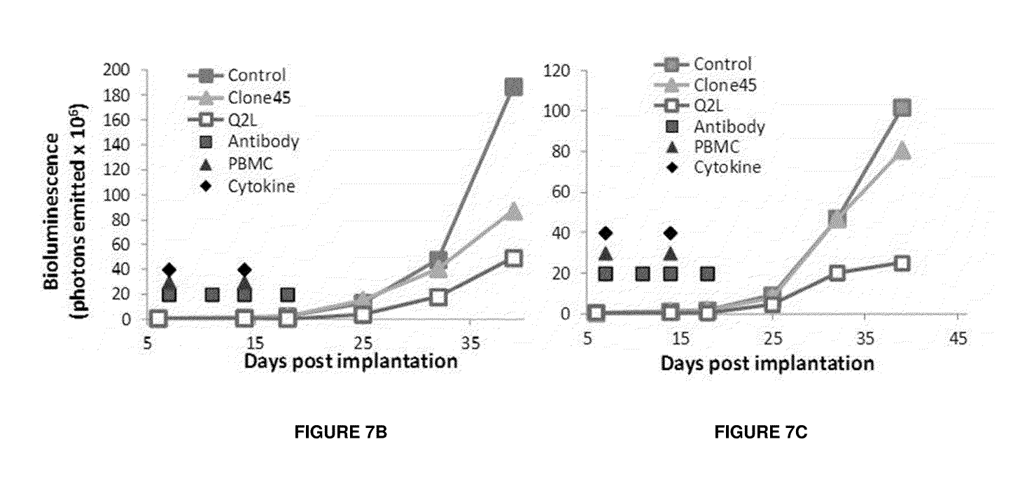

Q2L scFv-Fc was next tested for itsanti-tumor effect in vivo in DKO mice xenografted intravenously 7 days prior with BV173 acute lymphoblastic leukemia (ALL) cells. In the first tumor model, four i.v. injections of Q2L suppressed s.c. tumor growth, but not when control scFv-Fc was used; anti-tumor effect was observed even without the infusion of human effectors (FIG. 7A). However, tumor growth suppression was transient; three weeks later the leukemia regrew (data not shown).

In the second tumor model, human effector cells and cytokine were added. Injection of effectors along with four low doses of Q2L nearly eliminated the leukemia in comparison to treatments with effectors alone (FIG. 7B). Cytokine IL-15 injection was used to augment the ADCC activity of Q2L. As expected, the leukemia rapidly disseminated in the body with no activity by Clone45 in comparison to Q2L-treated mice (FIG. 7B). Q2L also significantly improved survival (data not shown). These results suggest that the higher affinity of Q2L translated into a significantly enhanced anti-tumor effect.

### DISCUSSION

The affinity maturation of Clone45 was carried out using complementary technologies: yeast display and in silico computation. The yeast-display library was initially generated based on scFv Clone45 where the CDR residues were randomized and clones selected for enhanced binding to WT1₁₂₆/HLA-A2 but not to irrelevant complexes. Using a minimal 20-fold to a maximal 100-fold affinity improvement boundaries, 3 clones were selected. Using homology modeling, the simulated structure of scFv recognizing the HLA-A2-WT1₁₂₆ complex was used to identify the two key residues responsible for interaction with the peptide motif, while residues facing the MHC helices were left unchanged. The final mutant, Q2L with two mutations in the CDR2 regions of the heavy and light chains, achieved a 100-fold improvement in affinity. It was noteworthy that the picomolar K_{D} (2 pM) of Q2L as bivalent scFv-Fc was the highest among reported TCR-like antibodies (9.9 to 294 nM) (Sergeeva, A., et al., Blood, 2011. 117(16): p. 4262-72), and the only published anti-WT1/HLA-A2 antibody showed an affinity of 100 pM (Dao, T., et al., Sci Transl Med, 2013. 5(176): p. 176ra33). Q2L, with its long retention time of Q2L (slow k_{off}=7.18×10⁻² S⁻¹) displayed increased binding compared to the parental Clone45 (k_{off}=3.55×10⁻⁴S⁻¹), and in addition, Q2L was also more efficacious in cytotoxicity.

It was confirmed that Q2L, and not the parental Clone45 antibody, mediated efficient ADCC in vitro, and anti-tumor effect in vivo, a direct result of the affinity maturation. The studies showed that the addition of human effectors and cytokine could enhance the antibody effects and extend survival, most likely through Fc-receptor dependent ADCC mechanisms in the presence of human NK cells and myeloid cells. In order to exploit cytotoxic T cells, genetic modifications using CARs seem to hold great promise (Cheung, N. K. and M. A. Dyer, Nat Rev Cancer, 2013. 13(6): p. 397-411). While conventional CARs target cell surface proteins and are not restricted to a particular HLA, there are theoretical advantages of using a CAR directed at the class I MHC peptide complex, since all internal proteins are potentially visible through this window. The affinity matured Q2L enabled CAR-modified T cells displayed potent cytolytic capacity in vitro against AML and breast cancer cell lines. Additionally, it was demonstrated that Q2L-CAR T cells recognized tumor cells in a WT1-dependent fashion. In contrast, no lysis was observed for parental Clone45 in the same format.

Natural killer (NK) cells are part of the innate immune system and the body's first line of defense against viral infection and malignance (Esser, R., et al., J Cell Mol Med, 2012. 16(3): p. 569-81). Unlike T cells expressing the TCR, NK cells are devoid of receptors for common tumor antigens (Kruschinski, A., et al., Proc Natl Acad Sci USA, 2008. 105(45): p. 17481-6). In addition, unlike transformed cells of hematopoietic origin which express NK activation ligands, solid tumors are relatively resistant to NK killing (Kruschinski, A., et al., Proc Natl Acad Sci USA, 2008. 105(45): p. 17481-6). In fact, most neuroblastoma cells were resistant to NK cells (Cho, D., et al., Clin Cancer Res, 2010. 16(15): p. 3901-9) and in studies refractory to parental NK-92-MI cells (data not shown). However, they were effectively lysed by Q2L-CAR NK-92-MI even when their HLA expression was low. Whether CAR-modified NK cells could overcome resistance mechanisms of neuroblastoma will require testing of patient NK cells and their tumor samples. Since the NK-92-MI cell line was safe in adoptive cancer immunotherapy (Esser, R., et al., J Cell Mol Med, 2012. 16(3): p. 569-81), Q2L-CAR modified NK-92-MI cell could be a therapeutic agent for WT1-expressing tumors. As patient-derived NK cells are engineered and expanded more and more efficiently, Q2L-CAR modified NK cells may be another therapeutic alternative (Shook, D. R. and D. Campana, Tissue Antigens, 2011. 78(6): p. 409-15).

Overall, a facile strategy for generating TCR-like antibodies with picomolar affinity and high specificity was developed. The results suggested that Q2L might be developed against leukemia- and solid tumor-specific WT1/HLA-A2 complexes. Such antibodies are critical diagnostic tools to study specific peptide expression in fresh tumors, as a biomarker of immunotherapy directed against the peptide or the antigen, and provide a sensitive and specific tool to study the biology of these tumor-associated peptides. When the peptide is derived from tumor-associated antigens (e.g. WT1) or viral antigens (Tassev, D. V., M. Cheng, and N. K. Cheung, Cancer Gene Ther, 2012. 19(2): p. 84-100) they have the potential to provide a sensitive and specific probe to detect or to isolate circulating tumor cells in patients. As monoclonal therapeutics directed against peptides or antigens expressed by the most common human cancers (Cheever, M. A., et al., Clin Cancer Res, 2009. 15(17): p. 5323-37), they greatly expand the possibilities of clinical application of TCR-like antibodies).

Although the foregoing invention has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims. All references cited herein are hereby incorporated in their entirety into the present application.

1. An isolated antigen-binding protein or fragment or derivative thereof comprising one of: (A) an antigen-binding region comprising an amino acid sequence selected from the group consisting of SEQ ID NOs: 2 to 8 or 27; or (B) an antigen-binding region comprising a heavy chain variable domain (VH) and a light chain variable domain (VL), wherein the VH and VL, respectively, comprise amino acid sequences selected from the group consisting of SEQ ID NOs: (i) 9 and 13; (ii) 9 and 15; (iii) 11 and 16; (iv) 9 and 17; (v) 12 and 14; (vi) 9 and 14; and (vii) 10 and 13; or (C) an antigen-binding region comprising: (i) the following three heavy chain (HC) CDRs: (a) a HC CDR1 comprising the amino acid sequence set forth in SEQ ID NO: 18; and (b) a HC CDR2 comprising an amino acid sequence selected from the group consisting of SEQ ID NOs: 19 and 20; and (c) a HC CDR3 comprising the amino acid sequence set forth in SEQ ID NO: 21; and (ii) the following three light chain (LC) complementarity determining regions (CDRs): (a) a LC CDR1 comprising an amino acid sequence selected from the group consisting of SEQ ID NOs: 22 and 23; and (b) a LC CDR2 comprising an amino acid sequence selected from the group consisting of SEQ ID NOs: 24 and 25; and (c) a LC CDR3 comprising the amino acid sequence set forth in SEQ ID NO: 26.

2. The isolated antigen-binding protein or fragment or derivative thereof of claim 1 comprising one of: (A) an antigen-binding region comprising the amino acid sequence set forth in SEQ ID NO: 2; or (B) an antigen-binding region comprising a heavy chain variable domain (VH) and a light chain variable domain (VL), wherein the VH and VL, respectively, comprise the amino acid sequences SEQ ID NOs: 9 and 13; or (C) an antigen-binding region comprising: (i) the following three heavy chain (HC) complementarity determining regions (CDRs): (a) a HC CDR1 comprising the amino acid sequence set forth in SEQ ID NO: 18; and (b) a HC CDR2 comprising the amino acid sequence set forth in SEQ ID NO: 20; and (c) a HC CDR3 comprising the amino acid sequence set forth in SEQ ID NO: 21; and (ii) the following three light chain (LC) CDRs: (a) a LC CDR1 comprising the amino acid sequence set forth in SEQ ID NO: 22; and (b) a LC CDR2 comprising the amino acid sequence set forth in SEQ ID NO: 25; and (c) a LC CDR3 comprising the amino acid sequence set forth in SEQ ID NO: 26.

3. The isolated antigen-binding protein or fragment or derivative thereof having a heavy chain variable region comprising CDR1, CDR2 and CDR3 from SEQ ID NO: 9, and a light chain variable region comprising CDR1, CDR2 and CDR3 from SEQ ID NO: 13.

4. The isolated antigen-binding protein or fragment or derivative thereof of claim 1, wherein the light chain variable region is at least 90% identical to SEQ ID NO: 9, and the heavy chain variable region is at least 90% identical to SEQ ID NO: 13; and wherein the antigen-binding protein is not Clone45.

5. (canceled)

6. The isolated antigen-binding protein or fragment or derivative thereof of claim 1, wherein the isolated antigen-binding protein or fragment or derivative thereof is a full-length antibody, a substantially intact antibody, a Fab fragment, a F(ab′)2 fragment, or a single chain variable fragment (scFv).

7. The isolated antigen-binding protein or fragment or derivative thereof of claim 6, wherein the antibody is a scFv.

8. The isolated antigen-binding protein or fragment or derivative thereof of claim 1, wherein the isolated antigen-binding protein or fragment or derivative thereof is a chimeric antigen receptor (CAR).

9. The scFV of claim 7, comprising a VH and a VL linked by an amino acid spacer, wherein the VH and VL, respectively, comprise amino acid sequences selected from the group consisting of SEQ ID NOs: (i) 9 and 13; (ii) 9 and 15; (iii) 11 and 16; (iv) 9 and 17; (v) 12 and 14; (vi) 9 and 14; and (vii) 10 and 13.

10. (canceled)

11. A fusion protein comprising the antigen-binding protein or fragment or derivative thereof of claim 1, wherein the fusion protein comprises a scFv-Fc fusion protein, immunoconjugate, or bispecific antibody.

12. The fusion protein of claim 11, wherein the fusion protein comprises the amino acid sequence set forth in SEQ ID NO: 27.

13. The fusion protein of claim 11, wherein the fusion protein comprises a bispecific antibody that engages T cells.

14. The fusion protein of claim 11, wherein the fusion protein comprises a second component selected from the group consisting of a cytotoxin, a detectable label, a radioisotope, a therapeutic agent, a liposome, a nanoparticle, a binding protein, or an antibody.

15. The fusion protein of claim 11, wherein the fusion protein is a scFv-Fc fusion protein comprising a Fc from human IgG1.

16. (canceled)

17. The isolated antigen-binding protein or fragment or derivative thereof of claim 1, wherein the antigen-binding protein or fragment or derivative thereof, scFv, or fusion protein specifically binds to an epitope on a HLA/peptide complex.

18. The isolated antigen-binding protein or fragment or derivative thereof of claim 17, wherein the peptide of the HLA/peptide complex comprises the amino acid sequence set forth in SEQ ID NO: 1 and the HLA of the HLA/peptide complex is a MHC class I molecule.

19. (canceled)

20. (canceled)

21. (canceled)

22. (canceled)

23. (canceled)

24. (canceled)

25. A nucleic acid encoding the isolated antigen-binding protein or fragment or derivative thereof of claim 1.

26. The nucleic acid of claim 25, wherein the nucleic acid encodes an isolated scFv comprising a VH and a VL linked by an amino acid spacer, wherein the VH and VL, respectively, comprise amino acid sequences selected from the group consisting of SEQ ID NOs: (i) 9 and 13; (ii) 9 and 15; (iii) 11 and 16; (iv) 9 and 17; (v) 12 and 14; (vi) 9 and 14; and (vii) 10 and 13.

27. An expression vector comprising the nucleic acid of claim 25.

28. A host cell transfected with the expression vector of claim 27.

29. The host cell of claim 28, wherein the host cell is a T-cell.

30. A pharmaceutical composition comprising an antigen-binding protein or fragment or derivative thereof of claim 1 and a physiologically acceptable diluent, excipient or carrier.

31. A cell expressing a chimeric antigen receptor (CAR) comprising the antigen-binding protein or fragment or derivative thereof of claim 1.

32. The cell of claim 31, wherein the cell is a T cell or natural killer (NK) cell.

33. A method of inhibiting tumor growth or metastasis comprising contacting a tumor cell with an effective amount of an antigen-binding protein or fragment or derivative thereof of claim 1.

34. (canceled)

35. (canceled)

36. A method of treatment comprising isolating T-cells from a subject, transfecting the T-cells with a vector comprising a nucleic acid encoding an isolated scFv comprising a VH and a VL linked by an amino acid spacer, wherein the VH and VL, respectively, comprise amino acid sequences selected from the group consisting of SEQ ID NOs: (i) 9 and 13; (ii) 9 and 15; (iii) 11 and 16; (iv) 9 and 17; (v) 12 and 14; (vi) 9 and 14; and (vii) 10 and 13, and administering the transfected T-cells to the subject.

37. (canceled)

38. (canceled)

39. (canceled)

40. (canceled)


SEQUENCE LISTING (39 sequences)

SEQ ID NO: 1 - protein, 9 aa, Homo sapiens
RMFPNAPYL

SEQ ID NO: 2 - protein, 241 aa, Artificial Sequence
  Synthetic peptide
EVQLLESGGGLVQPGGSLRLSCAASGFTFSSYAMSWVRQAPGKGLEWVSLIDPWGQETLYADSVKGRFTISRDNSKNTLYLQMNSLRAEDTAVYYCAKLTGRFDYWGQGTLVTVSSGGGGSGGGGSGGGGSTDIQMTQSPSSLSASVGDRVTITCRASQSISSYLNWYQQKPGKAPKLLIYSASLLQSGVPSRFSGSGSGTDFTLTISSLQPEDFATYYCQQGPGTPNTFGQGTKVEIKRA

SEQ ID NO: 3 - protein, 241 aa, Artificial Sequence
  Synthetic peptide
EVQLLESGGGLVQPGGSLRLSCAASGFTFSSYAMSWVRQAPGKGLEWVSLIDPWGQETLYADSVKGRFTISRDNSKNTLYLQMNSLRAEDTAVYYCAKLTGRFDYWGQGTLVTVSSGGGGSGGGGSGGGGSTNIQMTQSPSSLSASVGDRVTITCRASQSISSYLNWYQQKPGKAPKLLIYSASLLQSGVPSRFSGNGSGTDFTLTISSLQPEDFATYYCQQGPGTPNTFGQGTKVEIKRA

SEQ ID NO: 4 - protein, 241 aa, Artificial Sequence
  Synthetic peptide
EVQLLESGGGLVQPGGSLRLSCAASGFTFSSYAMSWVRQAPGKGLEWVSLIDPWGQETLYADSVKGRFTISRDNSKNTLYLQMNSLGAEDTAVYYCAKLTGRFDYWGQGTLVTVSSGGGGSGGGGSGGGGSTDIQMTQSPSSLSASVGDRVTITCRASQSISSYLNWYQQKPGKAPKLLIYSASQLQSGVPSRFSGSGSGTDFTLTISNLQPEDFATYYCQQGPGTPNTFGQGTKVEIKRA

SEQ ID NO: 5 - protein, 241 aa, Artificial Sequence
  Synthetic peptide
EVQLLESGGGLVQPGGSLRLSCAASGFTFSSYAMSWVRQAPGKGLEWVSLIDPWGQETLYADSVKGRFTISRDNSKNTLYLQMNSLRAEDTAVYYCAKLTGRFDYWGQGTLVTVSSGGGGSGGGGSGGGGSTDIQMTQSPSSLSASVGDRVTITCRASQSISNYLNWYQQKPGKAPKLLIYSASQLQSGVPSRFSGSGSGTVFTLTISSLQPEDFATYYCQQGPGTPNTFGQGTKVEIKRA

SEQ ID NO: 6 - protein, 241 aa, Artificial Sequence
  Synthetic peptide
EVQLLESGGGLVQPGGSLRLSCAASGFTFSSYAMSWVRQAPGKGLEWVSLIDPWGQETLYADSVKGRFTISRDNSKNTLYLQMNSLRAEDTAVYYCAKLTGRFDYWGQGTLVTVSSGGGGSGGGGSGGGGSTDIQMTQSPSSLSASVGDRVTITCRASQSISSYLNWYQQKPGKAPKLLIYSASQLQSGVPSRFSGSGSGTDFTLTISSLQPEDFATYYCQQGPGTPNTFGQGTKVEIKRA

SEQ ID NO: 7 - protein, 241 aa, Artificial Sequence
  Synthetic peptide
EVQLLESGGGLVQPGGSLRLSCAASGFTFSSYAMSWVRQAPGKGLEWVSQIDPWGQETLYADSVKGRFTISRDNSKNTLYLQMNSLRAEDTAVYYCAKLTGRFDYWGQGTLVTVSSGGGGSGGGGSGGGGSTDIQMTQSPSSLSASVGDRVTITCRASQSISSYLNWYQQKPGKAPKLLIYSASLLQSGVPSRFSGSGSGTDFTLTISSLQPEDFATYYCQQGPGTPNTFGQGTKVEIKRA

SEQ ID NO: 8 - protein, 241 aa, Artificial Sequence
  Synthetic peptide
EVQLLESGGGLVQPGGSLRLSCAASGFLFSSYAMSWVRQAPGKGLEWVSLIDPWGQETLYADSVKGRFTISRDNSKNTLYLQMNSLRAEDTAVYYCAKLTGRFDYWGQGTLVTVSSGGGGSGGGGSGGGGSTDIQMTQSPSSLSASVGDRVTITCRASQSISSYLNWYQQKPGKAPKLLIYSASQLQSGVPSRFSGSGSGTDFTLTISSLQPEDFATYYCQQGPGTPNTFGQGTKVEIKRA

SEQ ID NO: 9 - protein, 116 aa, Artificial Sequence
  Synthetic peptide
EVQLLESGGGLVQPGGSLRLSCAASGFTFSSYAMSWVRQAPGKGLEWVSLIDPWGQETLYADSVKGRFTISRDNSKNTLYLQMNSLRAEDTAVYYCAKLTGRFDYWGQGTLVTVSS

SEQ ID NO: 10 - protein, 116 aa, Artificial Sequence
  Synthetic peptide
EVQLLESGGGLVQPGGSLRLSCAASGFTFSSYAMSWVRQAPGKGLEWVSQIDPWGQETLYADSVKGRFTISRDNSKNTLYLQMNSLRAEDTAVYYCAKLTGRFDYWGQGTLVTVSS

SEQ ID NO: 11 - protein, 116 aa, Artificial Sequence
  Synthetic peptide
EVQLLESGGGLVQPGGSLRLSCAASGFTFSSYAMSWVRQAPGKGLEWVSLIDPWGQETLYADSVKGRFTISRDNSKNTLYLQMNSLGAEDTAVYYCAKLTGRFDYWGQGTLVTVSS

SEQ ID NO: 12 - protein, 116 aa, Artificial Sequence
  Synthetic peptide
EVQLLESGGGLVQPGGSLRLSCAASGFLFSSYAMSWVRQAPGKGLEWVSLIDPWGQETLYADSVKGRFTISRDNSKNTLYLQMNSLRAEDTAVYYCAKLTGRFDYWGQGTLVTVSS

SEQ ID NO: 13 - protein, 109 aa, Artificial Sequence
  Synthetic peptide
DIQMTQSPSSLSASVGDRVTITCRASQSISSYLNWYQQKPGKAPKLLIYSASLLQSGVPSRFSGSGSGTDFTLTISSLQPEDFATYYCQQGPGTPNTFGQGTKVEIKRA

SEQ ID NO: 14 - protein, 109 aa, Artificial Sequence
  Synthetic peptide
DIQMTQSPSSLSASVGDRVTITCRASQSISSYLNWYQQKPGKAPKLLIYSASQLQSGVPSRFSGSGSGTDFTLTISSLQPEDFATYYCQQGPGTPNTFGQGTKVEIKRA

SEQ ID NO: 15 - protein, 109 aa, Artificial Sequence
  Synthetic peptide
NIQMTQSPSSLSASVGDRVTITCRASQSISSYLNWYQQKPGKAPKLLIYSASLLQSGVPSRFSGNGSGTDFTLTISSLQPEDFATYYCQQGPGTPNTFGQGTKVEIKRA

SEQ ID NO: 16 - protein, 109 aa, Artificial Sequence
  Synthetic peptide
DIQMTQSPSSLSASVGDRVTITCRASQSISSYLNWYQQKPGKAPKLLIYSASQLQSGVPSRFSGSGSGTDFTLTISNLQPEDFATYYCQQGPGTPNTFGQGTKVEIKRA

SEQ ID NO: 17 - protein, 109 aa, Artificial Sequence
  Synthetic peptide
DIQMTQSPSSLSASVGDRVTITCRASQSISNYLNWYQQKPGKAPKLLIYSASQLQSGVPSRFSGSGSGTVFTLTISSLQPEDFATYYCQQGPGTPNTFGQGTKVEIKRA

SEQ ID NO: 18 - protein, 5 aa, Artificial Sequence
  Synthetic peptide
SYAMS

SEQ ID NO: 19 - protein, 17 aa, Artificial Sequence
  Synthetic peptide
QIDPWGQETLYADSVKG

SEQ ID NO: 20 - protein, 17 aa, Artificial Sequence
  Synthetic peptide
LIDPWGQETLYADSVKG

SEQ ID NO: 21 - protein, 7 aa, Artificial Sequence
  Synthetic peptide
LTGRFDY

SEQ ID NO: 22 - protein, 11 aa, Artificial Sequence
  Synthetic peptide
RASQSISSYLN

SEQ ID NO: 23 - protein, 11 aa, Artificial Sequence
  Synthetic peptide
RASQSISNYLN

SEQ ID NO: 24 - protein, 7 aa, Artificial Sequence
  Synthetic peptide
SASQLQS

SEQ ID NO: 25 - protein, 7 aa, Artificial Sequence
  Synthetic peptide
SASLLQS

SEQ ID NO: 26 - protein, 9 aa, Artificial Sequence
  Synthetic peptide
QQGPGTPNT

SEQ ID NO: 27 - protein, 470 aa, Artificial Sequence
  Synthetic peptide
EVQLLESGGGLVQPGGSLRLSCAASGFTFSSYAMSWVRQAPGKGLEWVSLIDPWGQETLYADSVKGRFTISRDNSKNTLYLQMNSLRAEDTAVYYCAKLTGRFDYWGQGTLVTVSSGGGGSGGGGSGGGGSTDIQMTQSPSSLSASVGDRVTITCRASQSISSYLNWYQQKPGKAPKLLIYSASLLQSGVPSRFSGSGSGTDFTLTISSLQPEDFATYYCQQGPGTPNTFGQGTKVEIKRKGPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 9 aa, Artificial Sequence
  Synthetic peptide
NLVPMVATV

SEQ ID NO: 29 - protein, 9 aa, Artificial Sequence
  Synthetic peptide
RIITSTILV

SEQ ID NO: 30 - protein, 9 aa, Artificial Sequence
  Synthetic peptide
LLEEMFLTV

SEQ ID NO: 31 - protein, 9 aa, Artificial Sequence
  Synthetic peptide
SLGEQQYSV

SEQ ID NO: 32 - protein, 9 aa, Artificial Sequence
  Synthetic peptide
CMTWNQMNL

SEQ ID NO: 33 - protein, 9 aa, Artificial Sequence
  Synthetic peptide
LMLGEFLKL

SEQ ID NO: 34 - protein, 10 aa, Artificial Sequence
  Synthetic peptide
FLTPKKLQCV

SEQ ID NO: 35 - DNA, 21 bp, Artificial Sequence
  Synthetic polynucleotide
tcagttttggcccaggcggcc

SEQ ID NO: 36 - DNA, 21 bp, Artificial Sequence
  Synthetic polynucleotide
accactagttgggccggcctg

SEQ ID NO: 37 - DNA, 54 bp, Artificial Sequence
  Synthetic polynucleotide
cttcgctgtttttcaatattttctgttattgcttcagttttggcccaggcggcc

SEQ ID NO: 38 - DNA, 56 bp, Artificial Sequence
  Synthetic polynucleotide
gagccgccaccctcagaaccgccaccctcagagccaccactagttgggccggcctg

SEQ ID NO: 39 - protein, 10 aa, Artificial Sequence
  Synthetic peptide
ELMLGEFLKL